(19) United States
(12) Patent Application Publication
(10) Pub. No.: US 2026/0134951 A1
(43) Pub. Date: May 14, 2026

(54) TRAINING AND UTILIZING NEURAL NETWORK POTENTIAL MODELS HAVING A MULTI-TASK ARCHITECTURE TO GENERATE QUANTUM MECHANICS PROPERTY PREDICTIONS

(71) Applicant: Recursion Pharmaceuticals, Inc., Salt Lake City, UT (US)

(72) Inventors: Cristian GABELLINI, Montreal (CA); Prudencio Murphy Akouété TOSSOU, Montreal (CA); Stephan THALER, Montreal (CA)

(21) Appl. No.: 18/913,456

(22) Filed: Oct. 11, 2024

(51) Int. Cl. G16C 10/00; G06N 3/08; G16C 20/30; G16C 20/70

(52) U.S. Cl. CPC G16C 10/00; G06N 3/08; G16C 20/30; G16C 20/70

(57) ABSTRACT

The present disclosure relates to systems, non-transitory computer-readable media, and methods for training and utilizing neural network potential models to generate quantum mechanics property predictions of different classes from training compound geometries and/or query compound geometries. For example, the disclosed systems can utilize a backbone neural network to generate a latent feature representation of a training compound geometry and/or a query compound geometry. Moreover, the disclosed systems can utilize different task heads to generate a quantum mechanics property prediction from different classes from the latent feature representation. Further, the disclosed systems can train neural network potential models based on ground truth quantum mechanics property predictions from the different classes to improve functionality of implementing computers.

### BACKGROUND

Recent years have seen significant developments in hardware and software platforms for training and utilizing machine learning models in conjunction with computer-implemented pharmaceutical discovery systems. For example, conventional systems utilize large volumes of training data to analyze chemical compounds and generate various molecular dynamics predictions. Despite these recent advances, conventional systems suffer from a number of technical deficiencies, particularly with regard to efficiency, accuracy, and operational inflexibility in implementing machine learning technologies. These deficiencies are particularly profound with regard to computational resources required to train new models.

### SUMMARY

Embodiments of the present disclosure provide benefits and/or solve one or more of the foregoing or other problems in the art with systems, non-transitory computer-readable media, and methods for training neural network potential models utilizing a multi-task architecture to generate quantum mechanics property predictions for utilization in molecular dynamics simulations of compound geometries. For example, the disclosed systems can utilize a backbone neural network of a neural network potential model to generate feature representations of compound geometries. The disclosed systems can then utilize different prediction heads to generate different quantum mechanics property predictions from the feature representations. The disclosed systems can compare the quantum mechanics property predictions from the different prediction heads with ground truths that correspond to different quantum mechanics classes (e.g., ground truth quantum mechanics property predictions generated from different approaches having different fidelities). The disclosed systems can then update parameters of the neural network potential model to increase the accuracy of the neural network potential model.

For example, the disclosed systems can utilize high fidelity training data to train a first prediction head to generate high fidelity quantum mechanics property predictions from the feature representations. In addition, the disclosed systems can utilize low fidelity training data to train a second prediction head to generate low fidelity quantum mechanics property predictions from the feature representations. Indeed, in some embodiments, the disclosed systems can utilize training data of different fidelities to train prediction heads to generate quantum mechanics property predictions of corresponding qualities. The disclosed systems can compare the quantum mechanics property predictions with ground truths of corresponding quantum mechanics classes and update the parameters of the neural network potential model according to the comparison. By utilizing different quantum mechanics classes to train different prediction heads and a backbone model, the disclosed systems can more efficiently leverage high fidelity and low fidelity training data in building an accurate neural network potential model.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates an implicit delta learning system utilizing a neural network potential model to generate quantum mechanics property predictions from compound geometries in accordance with one or more embodiments.

FIG. 2 illustrates an example architecture of a neural network potential model in accordance with one or more embodiments.

FIG. 3A illustrates the implicit delta learning system generating a high fidelity property prediction and a low fidelity property prediction from a feature representation of a training compound geometry in accordance with one or more embodiments.

FIG. 3B illustrates the implicit delta learning system generating a high fidelity property prediction from a first feature representation of a first training compound geometry and generating a low fidelity property prediction from a second feature representation of a second training compound geometry in accordance with one or more embodiments.

FIG. 4A illustrates an additional architecture of a neural network potential model in accordance with one or more embodiments.

FIG. 4B illustrates an additional architecture of a neural network potential model in accordance with one or more embodiments.

FIGS. 5A-5E illustrate experimental results achieved by the implicit delta learning system in accordance with one or more embodiments.

FIG. 6 illustrates an example environment of the implicit delta learning system in accordance with one or more embodiments.

FIG. 7 illustrates an example series of acts for training a neural network potential model in accordance with one or more embodiments.

FIG. 8 illustrates a block diagram of a computing device for implementing one or more embodiments.

### DETAILED DESCRIPTION

This disclosure describes one or more embodiments of an implicit delta learning system 100 that trains a neural network potential model (NNP) having a multi-task architecture to generate quantum mechanics property predictions from compound geometries. For example, in one or more embodiments, the implicit delta learning system 100 can utilize a neural network backbone of an NNP to generate feature representations from compound geometries. The implicit delta learning system 100 can train prediction heads of an NNP to generate different classes of quantum mechanics property predictions from the feature representations. Additionally, the implicit delta learning system 100 can compare the different classes of quantum mechanics property predictions with corresponding ground truths from different quantum mechanics representation classes (e.g., ground truth quantum mechanics property predictions of different quantum mechanics representation classes) and modify parameters of the NNP according to the comparisons.

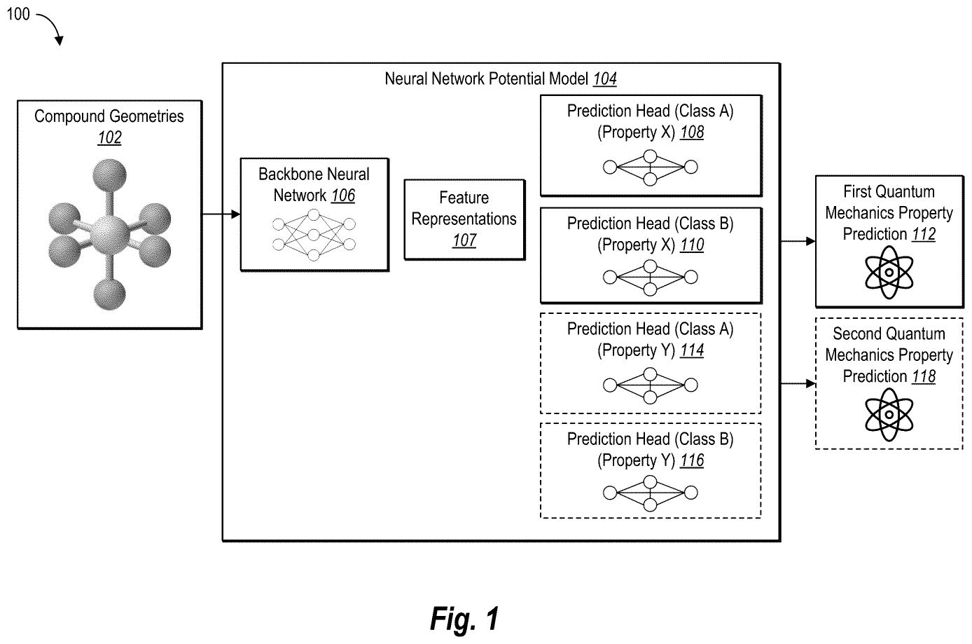

As mentioned above, the implicit delta learning system 100 can generate quantum mechanics property predictions from feature representations of compound geometries. As illustrated in FIG. 1, the implicit delta learning system 100 receives compound geometries 102. The implicit delta learning system 100 can access or otherwise retrieve the compound geometries 102 from a database (e.g., the Materials Project, the Open Quantum Materials Database, or the Quantum Machine Learning database, among others). Additionally or alternatively, the implicit delta learning system 100 can generate the compound geometries 102 or interface with third-party software to cause the compound geometries 102 to be generated.

As illustrated in FIG. 1, the implicit delta learning system 100 utilizes a neural network potential model 104 (“NNP 104”). The NNP 104 can include a backbone neural network 106. Indeed, the implicit delta learning system 100 can utilize the backbone neural network 106 of the NNP 104 to generate feature representations 107 from the compound geometries 102. Additionally, the NNP 104 can include multiple prediction heads of multiple classes. Moreover, the NNP 104 can include multiple prediction heads of a single class that generate different quantum mechanics property predictions according to their respective class. For example the implicit delta learning system 100 can include a prediction head (class A) (property X) 108, a prediction head (class B) (Property X) 110, a prediction head (class A) (property Y) 114, and a prediction head (class B) (property Y) 116. The implicit delta learning system 100 can utilize the prediction heads of the NNP 104 to generate quantum mechanics property predictions, such as the first quantum mechanics property prediction 112 and the second quantum mechanics property prediction 118 . . .

Indeed, as illustrated, the implicit delta learning system 100 can train each of the multiple prediction heads to generate quantum mechanics property predictions of different classes and quality levels. For example, the implicit delta learning system 100 can train the prediction head (class A) (property X) 108 to generate quantum mechanics property predictions of a quality level of class A (e.g., where class A indicates high fidelity/high quality property predictions) for property X (e.g., where property X indicates a specific quantum mechanics property, such as, for example, electron density). Moreover, the implicit delta learning system 100 can train the prediction head (class B) (property X) 110 to generate quantum mechanics property predictions of a quality level of class B (e.g., where class B indicates low fidelity/low quality property predictions) for property X. Additionally, the implicit delta learning system 100 can train the prediction head (class A) (property Y) 114 to generate high fidelity quantum mechanics property predictions for property Y (e.g., where property Y indicates a specific quantum mechanics property that is different from property X, such as molecular orbitals). Further, the implicit delta learning system 100 can train the prediction head (class B) (property Y) to generate low fidelity quantum mechanics property predictions for property Y.

Moreover, in some embodiments, the implicit delta learning system 100 can train prediction heads to generate quantum mechanics property predictions of other classes (e.g., qualities). For example, in some embodiments, the implicit delta learning system 100 can train prediction head B (class C) to generate quantum mechanics property predictions of a quality level of class C (e.g., where class B indicates medium fidelity/medium fidelity property predictions). Moreover, the implicit delta learning system 100 can train multiple prediction heads to generate quantum mechanics property predictions for different quantum mechanics properties, each of a quality level of class C. The implicit delta learning system 100 can train the NNP 104 by comparing the quantum mechanics property predictions of each class with a ground truth of a corresponding class to determine a measure of loss. Moreover, the implicit delta learning system 100 can compare the quantum mechanics property predictions of each prediction head with each other to determine a measure of loss due to the different classes/quality levels of the quantum mechanics property prediction.

As illustrated, the implicit delta learning system 100 can cause the NNP 104 generate a first quantum mechanics property prediction 112 for the compound geometries 102. Indeed, the implicit delta learning system 100 can utilize the prediction head (class A) (property X) 108 to generate a first quantum mechanics property prediction 112 of class A (e.g., a high fidelity/high quality quantum mechanics property prediction for the compound geometries 102). Indeed, by training the prediction head (class A) (property X) 108 based on ground truth quantum mechanics property predictions from class A, the implicit delta learning system 100 can then utilize the prediction head (class A) (property X) 108 to generate the first quantum mechanics property prediction 112 having a similar level of fidelity. Notably, however, the implicit delta learning system 100 trains the backbone neural network 106 based on predictions and ground truths for a variety of different prediction heads and corresponding classes and, in some embodiments, of corresponding quantum mechanics properties. Thus, the neural network potential model 104 improves in accuracy and performance by learning from ground truth quantum mechanics property predictions across a variety of different classes. This approach improves overall performance in generating predicted quantum mechanics property predictions at inference time for a particular prediction class (e.g., for generating predictions for a high-fidelity quantum mechanics representation class).

In addition, the implicit delta learning system 100 can utilize the first quantum mechanics property prediction 112 in a variety of downstream applications. For example, the implicit delta learning system 100 can utilize the first quantum mechanics property prediction 112 in one or more additional computer-implemented models to generate bioactivity predictions. To illustrate, the implicit delta learning system 100 can utilize quantum mechanics property predictions in molecular dynamics simulations to determine in-silico interactions of molecular systems and their dynamics (e.g., how pharmaceutical compounds interact within molecular systems of the body). The implicit delta learning system 100 can also utilize the feature representations 107 to generate bioactivity predictions, such as by utilizing the feature representations 107 to determine in-silico interactions of molecular systems and their dynamics.

For example, in one or more embodiments, the implicit delta learning system 100 can utilize the first quantum mechanics property prediction 112 to generate biological activity predictions for the compound geometries 102. For example, the implicit delta learning system 100 can analyze features of the first quantum mechanics property prediction 112 to determine a likelihood that one or more of the compound geometries 102 can be developed into potential treatments for disease. For example, the implicit delta learning system 100 can utilize a quantum mechanics property prediction to model interactions between compounds and proteins. Similarly, the implicit delta learning system 100 can utilize the quantum mechanics property prediction as input to other machine learning models to generate relationship predictions (e.g., between compounds or between compounds and genes). Moreover, the implicit delta learning system 100 can utilize the quantum mechanics property prediction to generate binding predictions, ADMET predictions, liability predictions, etc. Additionally, the implicit delta learning system 100 can utilize the feature representations 107 to determine the likelihood that one or more of the compound geometries can be developed into potential treatments for disease. Moreover, the implicit delta learning system 100 can analyze the feature representations 107 utilizing other machine learning models to predict/model interactions between compounds and proteins, or generate binding predictions, ADMET predictions, or liability predictions, among others.

In some implementations, the implicit delta learning system 100 can initiate a compound program analysis based on the first quantum mechanics property prediction 112 and/or the feature representations 107. Indeed, the implicit delta learning system 100 can utilize the first quantum mechanics property prediction 112 and/or the feature representations 107 to identify an anchor compound or anchor gene from the compound geometries 102. Upon determination of the anchor compound or anchor gene, the implicit delta learning system 100 can determine a program rating for the anchor compound and/or the anchor gene. For example, the implicit delta learning system 100 can identify a protein that corresponds to a gene/disease of interest. The implicit delta learning system 100 can utilize the first quantum mechanics property prediction 112 and/or the feature representations 107 to generate binding metrics for compound geometry that indicate the likelihood of the compound geometry binding with or otherwise interacting with a target compound and/or molecule. The implicit delta learning system 100 can utilize the binding metric to determine the program rating (e.g., the implicit delta learning system 100 can determine a high binding metric for a compound geometry that indicates a high likelihood that the compound geometry will bind with a target compound, and subsequently generate a high program rating for the compound geometry).

Indeed, in some embodiments, the implicit delta learning system 100 can utilize the program rating to initiate a compound program analysis by initiating an industrial program generation (IPG) process. To illustrate, the implicit delta learning system 100 can utilize the IPG process to identify various components and/or requirements to develop the anchor compound into an advanced treatment for a disease. Specifically, the implicit delta learning system 100 can initiate the IPG process to identify information such as statistically strong connections in a biological map to patient-informed phenotypes, Trekseq confirmation (e.g., confirming anchor compound and anchor gene relationships utilizing transcriptomics), Structure-Activity Relationships (SAR) confidence, among others, moreover, the implicit delta learning system 100 can utilize the program rating to initiate an industrialized compound generation process (ICG) to apply steps subsequent to the IPG process. For example, the implicit delta learning system 100 can utilize the ICG process to test the anchor compound with various analytical tests (e.g., SAR screens), or to identify other potential compounds to the anchor compound for use in the treatment of the disease.

As mentioned briefly above, conventional systems suffer from a number of technical deficiencies with regard to implementing computing devices. For example, conventional systems are often inefficient. Indeed, conventional systems require training with large volumes of high-fidelity training data to be able to generate high fidelity quantum mechanics property predictions. Collecting and/or generating such high-fidelity training data and then training neural network potential models on this data is computationally expensive. Accordingly, conventional systems require significant time and computational resources to generate training data and to train NNPs.

Some conventional systems utilize a delta-learning approach that teaches models to predict an energy difference (or delta) between low-fidelity property predictions and high-fidelity property predictions. Although this approach can reduce the number of high-fidelity samples needed during training, it suffers from significant efficiency problems at inference time. Indeed, this approach significantly increases inference costs due to on-the-fly low-fidelity property prediction calculations that are then utilized with a trained model to generate subsequent delta predictions.

Moreover, conventional systems are operationally inflexible. As an initial matter, many conventional systems can only utilize a single type of training data in building prediction models. For example, many conventional systems cannot leverage low-fidelity data without undermining prediction accuracy. Thus, conventional systems are often rigidly limited to only utilizing high-fidelity training data, which exacerbates the efficiency problems discussed above.

In addition, conventional systems are also inflexible and inaccurate with regard to model generalization. Indeed, because conventional systems train with a limited sample size of high-fidelity data corresponding to a particular region of the chemical space, resulting models cannot accurately generate predictions outside of the training domain. Thus, accuracy of such models decreases significantly for compounds that are significantly different than high-fidelity training samples observed during training.

As suggested by the foregoing discussion, the implicit delta learning system 100 provides a variety of technical advantages relative to conventional systems. For example, the implicit delta learning system 100 can improve the efficiency of conventional computing systems. Indeed, as illustrated in FIG. 1, the implicit delta learning system 100 can utilize quantum mechanics property predictions from a variety of different classes (e.g., high fidelity quantum mechanics property predictions and low fidelity quantum mechanics property predictions) during training. Specifically, the implicit delta learning system 100 can utilize a backbone architecture with multiple different prediction heads corresponding to different fidelities. By utilizing a mixture of training data from different classes having different levels of fidelity for different trained heads, the implicit delta learning system 100 can train accurate NNPs with fewer high-fidelity samples. Thus, the implicit delta learning system 100 can reduce time and computational resources needed to generate high-fidelity samples and train NNPs.

Furthermore, the implicit delta learning system 100 also improves efficiency relative to conventional delta-learning approaches. For example, at inference time, the implicit delta learning system 100 can analyze a compound geometry and utilize a trained prediction head (e.g., a high-fidelity prediction head) to directly generate a high-fidelity quantum mechanics property prediction. Thus, the implicit delta learning system 100 avoids the time and computational resources associated with generating low-fidelity quantum mechanics property predictions at inference time and then generating a delta prediction.

In addition to the efficiency improvements, in some embodiments, the implicit delta learning system 100 improves the accuracy of conventional systems. Indeed, by utilizing a multi-task architecture, the implicit delta learning system 100 can learn the chemical feature space from a variety of different classes of quantum mechanics property predictions. Thus, the implicit delta learning system 100 can improve the accuracy of trained models and resulting quantum mechanics property predictions relative to the time and computational expense of training.

Moreover, the implicit delta learning system 100 increases the operational flexibility of conventional systems. In contrast to conventional systems, the implicit delta learning system 100 can utilize a variety of different training data classes in building NNPs. Specifically, the implicit delta learning system 100 can utilize a flexible architecture of multiple different prediction heads that can accommodate different ground truth training data of different quantum mechanics representation classes. This improved flexibility leads to improved efficiency and accuracy, as mentioned above.

In addition, the implicit delta learning system 100 improves flexibility generalizing to a broader range of chemical feature space. Indeed, because the implicit delta learning system 100 can accommodate a variety of different classes of training data, it learns an increased spectrum of chemical feature space during training and learns to generate predictions across a wider range of compound geometries. This flexibility also leads to improved performance outside of the training domain in applying NNPs. Indeed, the implicit delta learning system 100 can more accurately generate quantum mechanics property predictions outside of the training domain relative to many conventional systems.

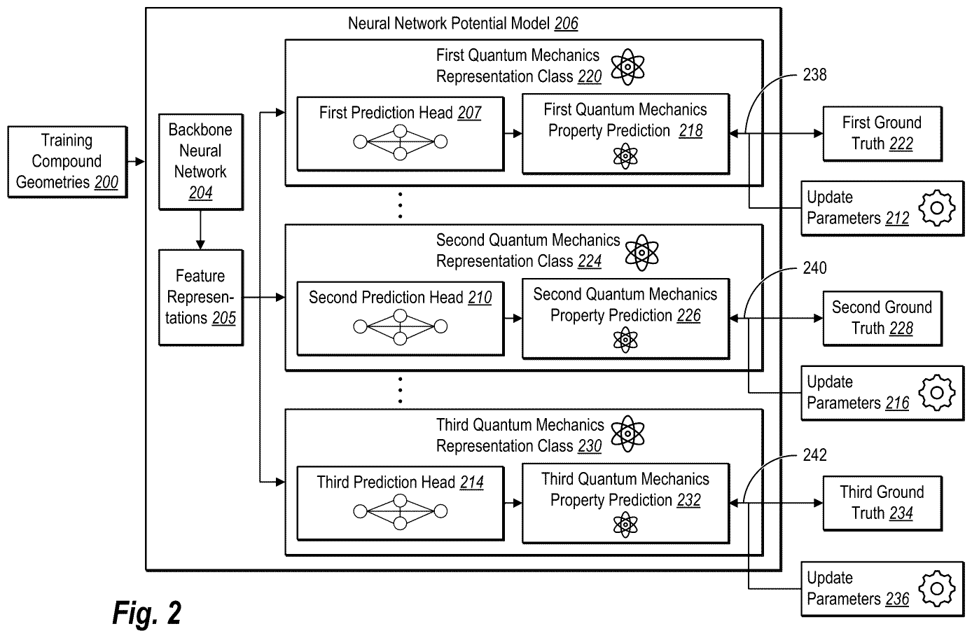

As previously mentioned, the implicit delta learning system 100 can train prediction heads of a neural network potential model to generate quantum mechanics property predictions of different classes. FIG. 2 illustrates the implicit delta learning system 100 utilizing prediction heads to generate quantum mechanics property predictions; comparing the quantum mechanics property predictions with ground truths of a first quantum mechanics representation class, a second quantum mechanics representation class, and a third quantum mechanics representation class; and updating parameters of the prediction heads of the neural network potential model.

As illustrated in FIG. 2, the implicit delta learning system 100 can provide training compound geometries 200 to a neural network potential model 206 (“NNP 206”). As used herein, the phrase “training compound geometries” refers to a training data set including information about chemical compounds. For example, the training data set can include various features or information regarding a compound geometry. To illustrate training data can include atomic configurations, atomic types within the atomic configurations, atomic positions (e.g., coordinates) within the atomic configurations, atomic connectivity (e.g., connections/bond types between atoms) within the atomic configurations, potential energy of atomic configurations, forces acting on atoms within the atomic configurations, or additional properties of the atomic configurations, among others.

Moreover, as used herein, the term “neural network potential model” refers to a machine learning model utilized to model the energy of a molecular system. In particular, a neural network potential model includes a neural network utilized to model the potential energy surface of a molecular system by predicting potential energy and forces acting on atoms within a molecule (e.g., based on their position).

As used herein, the term “machine learning model” includes a computer algorithm or a collection of computer algorithms that can be trained and or tuned based on inputs to approximate unknown functions. For example, a machine learning model can include a computer algorithm with branches, weights, or parameters that change based on training data to improve for a particular task. Thus, a machine learning model can utilize one or more learning techniques (e.g., supervised or unsupervised learning) to improve in accuracy and/or effectiveness. Example machine learning models include various types of decision trees (e.g., gradient boost models), support vector machines, Bayesian networks, random forest models, or neural networks (e.g., deep neural networks, generative adversarial neural networks, convolutional neural networks, recurrent neural networks, or diffusion neural networks). Similarly, as used herein, a neural network refers to a machine learning model of interconnected nodes (or neurons) organized into layers. A neural network can include parameters or weights between neurons that are adjusted during training to minimize the error (or measure of loss) in generating predictions.

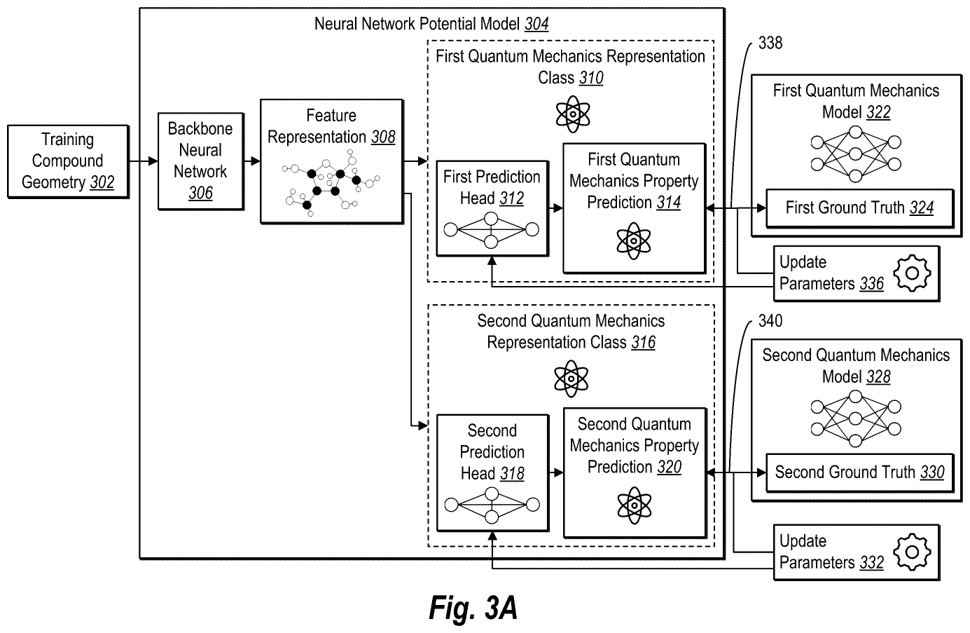

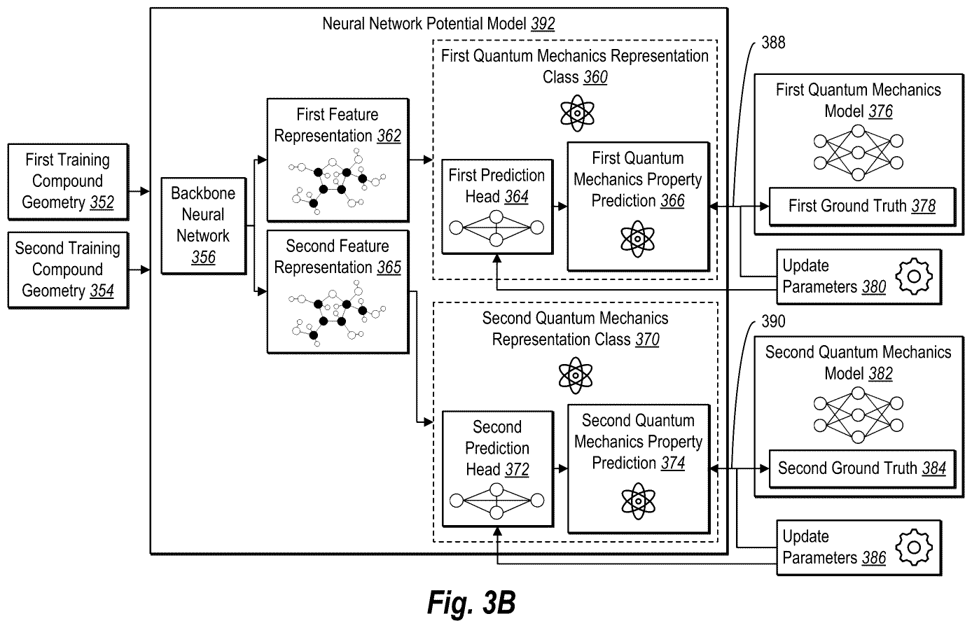

As illustrated in FIG. 2, the implicit delta learning system 100 can utilize a backbone neural network 204 of the NNP 206 to analyze the training compound geometries 200. For example, the backbone neural network 204 analyzes input features of the training compound geometry (e.g., a conformation of a molecule such as atoms, positions, and/or atomic numbers). Moreover, the backbone neural network 204 generates, from these input features, feature representations 205 of the training compound geometries. More information regarding the backbone neural network 204 is provided below (e.g., with regards to FIGS. 3A-3B).

As illustrated in FIG. 2, the implicit delta learning system 100 can utilize the backbone neural network 204 to generate feature representations 205 of the training compound geometries 200 and further analyze these feature representations 205 utilizing a plurality of prediction heads. Indeed, the NNP 206 can include several different branches corresponding to different classes of quantum mechanics property predictions, as well as different prediction heads within the different branches corresponding to specific types of quantum mechanics property predictions within the respective class. For example, the implicit delta learning system 100 can train different branches/subcomponents of the NNP 206 to generate quantum mechanics property predictions of different levels of quality/fidelity.

Indeed, as illustrated, the NNP 206 includes a first quantum mechanics representation class 220, a second quantum mechanics representation class 224, and a third quantum mechanics representation class 230. As used herein, the term “quantum mechanics representation class” refers to a type, category, or classification of quantum mechanics property predictions. In particular, a quantum mechanics representation class can include a representation and/or property prediction of energy levels of a compound generated by a particular method or model. This different quantum mechanics property predictions can include different quantum mechanical properties (potential energy levels, electron densities, molecular orbitals, etc.) for a compound generated by different computer-implemented algorithms having different levels of fidelity/quality/accuracy.

To illustrate, the first quantum mechanics representation class 220 can be derived from high-accuracy quantum mechanical calculations such as density functional theory (DFT). Similarly, a quantum mechanics representation class can include quantum mechanics property predictions generated utilizing other approaches, including, post-Hartree-Fock, Quantum Monte Carlo, Variational Monte Carlo, Diffusion Monte Carlo, Configuration Interaction, Full Configuration Interaction, Coupled Cluster with Single, Double, and Perturbative Triple Excitations, Density Matrix Renormalization Group, Complete Active Space Self-Consistent Field, Multireference Configuration Interaction, Multireference Perturbation Theory, Density Matrix Embedding Theory, among others. Thus, a first quantum mechanics property prediction includes refers a representation of a surface energy of a compound (e.g., a compound geometry or a training compound geometry) corresponding to a first quantum mechanics class.

Similarly, the implicit delta learning system 100 can utilize a “second quantum mechanics representation class that includes a category, group, or classification of quantum mechanics property predictions generated utilizes one or more alternative approaches (e.g., having a different level of fidelity/quality/accuracy). For example, the second quantum mechanics representation class 224 can be a set of low-fidelity representations and/or property predictions derived from methods such as Simplistic Basis Sets, Hartree-Fock Approximation, Hartree-Fock-based Semi-Empirical methods, Tight-Binding, Minimal Basis Set Density Functional Theory, or Extended Hückel Theory, among others. Compared to the high fidelity methods (e.g., the first quantum mechanics representation class 220), the low fidelity methods (e.g., the second quantum mechanics representation class 224) may have relatively lower accuracy (e.g., lower fidelity with regard to approximate electron correlation, approximations of wavefunctions, accounting for relativistic effects, etc.). Thus, a second quantum mechanics property prediction includes a representation of a surface energy of a compound (e.g., a compound geometry or a training compound geometry) within the second quantum mechanics class. (e.g., of a different class/quality than the first quantum mechanics property prediction).

Moreover, the implicit delta learning system 100 can also utilize a third quantum mechanics representation class or a different number of classes. For example, the implicit delta learning system 100 can utilize a third class corresponding to a medium fidelity (between a high-fidelity class and a low-fidelity class). For instance, the third quantum mechanics representation class can be more accurate than the low fidelity class (e.g., the second quantum mechanics representation class 224) but less accurate than the high fidelity class (e.g., the first quantum mechanics representation class 220). Indeed, a third quantum mechanics property prediction can be a representation and/or property prediction of the surface energy of a compound (e.g., a training compound geometry or a compound geometry) the implicit delta learning system 100 generates utilizing a third set of methods or computer-implemented models.

Returning to a discussion of FIG. 2, the implicit delta learning system 100 can cause the backbone neural network 204 to provide feature representations of the training compound geometries 200 to a first prediction head 207 (e.g., a prediction head that generates quantum mechanics property predictions of the first quantum mechanics representation class 220), a second prediction head 210 (e.g., a prediction head that generates quantum mechanics property predictions of the second quantum mechanics representation class 224), and a third prediction head 214 (e.g., a prediction head that generates quantum mechanics property predictions of the third quantum mechanics representation class 230).

As used herein, the term “prediction head” refers to a subcomponent of the NNP utilized to generate predictions. For example, a prediction head can include a machine learning component (such as a series of neural network layers) utilized to generate a prediction or output from a feature representation. In some implementations, a prediction head can include a multi-layer perceptron (“MLP”) with a single hidden layer.

As illustrated, the implicit delta learning system 100 can cause the first prediction head 207 to generate a first quantum mechanics property prediction 218, the second prediction head 210 to generate a second quantum mechanics property prediction 226, and the third prediction head 214 to generate a third quantum mechanics property prediction 232. In other words, the implicit delta learning system 100 can use the multi-task architecture of the neural network potential model 206 to generate multiple quantum mechanics property predictions.

As illustrated, the implicit delta learning system 100 can compare each quantum mechanics property prediction with a ground truth of a particular class/quality level. In particular, the implicit delta learning system 100 can utilize a loss function to compare quantum mechanics property predictions with different quantum mechanics property predictions from different quantum mechanics property classes to generate measures of loss. Moreover, the implicit delta learning system 100 can modify the parameters of each respective task head (e.g., the task head that created the quantum mechanics property prediction of the particular class/fidelity level) and the backbone neural network 204 according to the measure of loss. For example, the implicit delta learning system 100 can utilize back propagation and/or gradient descent to modify neural network parameters, reduce the measure of loss, and improve accuracy of the neural network potential model 206 over multiple training iterations.

For example, the implicit delta learning system 100 can perform an act 238 to compare the first quantum mechanics property prediction 218 with a first ground truth 222 (from the first quantum mechanics representation class 220) to generate a first measure of loss. The implicit delta learning system 100 can utilize the first measure of loss to perform an act 212 to modify parameters of the first prediction head 207 and the backbone neural network 204.

Moreover, the implicit delta learning system 100 can perform an act 240 to compare the second quantum mechanics property prediction 226 with a second ground truth 228 (from the second quantum mechanics representation class 224) to determine a second measure of loss. The implicit delta learning system 100 can utilize the second measure of loss to perform an act 216 to modify parameters of the second prediction head 210 and the backbone neural network 204.

Additionally, the implicit delta learning system 100 can perform an act 242 to compare the third quantum mechanics property prediction 232 with a third ground truth 234 (from the third quantum mechanics representation class 230) to determine a third measure of loss. The implicit delta learning system 100 utilize the third measure of loss to perform an act 236 to update parameters of the third prediction head 214 and the backbone neural network 204.

In this manner, the implicit delta learning system 100 trains each of the prediction heads to generate quantum mechanics property predictions corresponding to a particular quantum mechanics representation class. Specifically, the implicit delta learning system 100 trains the first prediction head 207 to generate quantum mechanics property predictions corresponding to the first quantum mechanics representation class 220, trains the second prediction heads 210 to generate quantum mechanics property predictions corresponding to second quantum mechanics representation class 224, and trains the third prediction head 214 to generate quantum mechanics property predictions corresponding to the third quantum mechanics representation class 230. Moreover, the implicit delta learning system 100 trains the backbone neural network 204 based on all of the various quantum mechanics representation classes. Thus, the implicit delta learning system 100 utilizes training samples from a variety of different sources to improve the backbone neural network 204 while utilizing particular prediction heads to generate various classes of predictions (e.g., including a high-fidelity prediction head for generating high-fidelity quantum mechanics property predictions at inference time).

In training a neural network potential model, the implicit delta learning system 100 can have a variety of different training data combinations. For example, in some implementations, the implicit delta learning system 100 identifies a training compound geometry with both high-fidelity and low-fidelity quantum mechanics property predictions. In some implementations, the implicit delta learning system 100 identifies some training compound geometries with high-fidelity quantum mechanics property predictions and some training compound geometries with low-fidelity quantum mechanics property predictions. In these different circumstances, the implicit delta learning system 100 can train and update parameters of a neural network potential model to generate accurate predictions.

Moreover, while FIG. 2 illustrates one prediction head per quantum mechanics representation class (e.g., the first prediction head 207 for the first quantum mechanics representation class 220, the second prediction head 210 for the second quantum mechanics representation class 224, and the third prediction head 214 for the third quantum mechanics representation class 230), as discussed above with regard to FIG. 1, the implicit delta learning system 100 can train the NNP 206 to include multiple prediction heads within a single quantum mechanics representation class. Indeed, the implicit delta learning system 100 can train a first plurality of prediction heads within the first quantum mechanics representation class 220, a second plurality of prediction heads within the second quantum mechanics representation class 224, and a third plurality of prediction heads within the third quantum mechanics representation class. Specifically, the implicit delta learning system 100 can train each prediction head of the first plurality of prediction heads to generate a high fidelity quantum mechanics property prediction for a different quantum mechanics property. Moreover, the implicit delta learning system 100 can train each prediction head of the second plurality of prediction heads to generate a low fidelity quantum mechanics property prediction for a different quantum mechanics property. Further, the implicit delta learning system 100 can train each prediction head of the third plurality of prediction heads to generate a medium fidelity quantum mechanics property prediction for a different quantum mechanics property.

Moreover, while FIG. 2 illustrates the implicit delta learning system 100 comparing the first ground truth 222 to the first quantum mechanics property prediction 218, the second ground truth 228 to the second quantum mechanics property prediction 226, and the third ground truth 234 to the third quantum mechanics property prediction 232, in some embodiments, the implicit delta learning system 100 can utilize a uniform ground truth to determine a uniform measure of loss by comparing the uniform ground truth to the first quantum mechanics property prediction 218, the second quantum mechanics property prediction 226, and the third quantum mechanics property prediction 232. Based on determining the uniform measure of loss, the implicit delta learning system 100 can update the parameters of the backbone neural network 204, the first prediction head 207, the second prediction head 210, and/or the third prediction head 214.

For example, FIG. 3A illustrates a circumstance where the implicit delta learning system 100 identifies a training compound geometry 302 having two ground truth quantum mechanics property predictions (e.g., a high-fidelity and low-fidelity quantum mechanics representation and/or property prediction) generated from different quantum mechanics models. Moreover, the implicit delta learning system 100 utilizes both of these quantum mechanics property predictions to train a neural network potential model 304.

As illustrated in FIG. 3A, the implicit delta learning system 100 can provide a training compound geometry 302 to a neural network potential model 304 (“NNP 304”). For example, the training compound geometry 302 can include one of the training compound geometries 200 of FIG. 2, and the NNP 304 can include the neural network potential model 206 of FIG. 2.

As illustrated, the NNP 304 can include a backbone neural network 306. As used herein, the term “backbone neural network” includes a neural network architecture that feeds information or data to multiple additional machine learning channels. For example, a backbone neural network can include a neural network that generates one or more feature representations that are utilized by different prediction heads.

The backbone neural network 306 can include a variety of neural network architectures. For example, the backbone neural network 306 can be a feedforward neural network (such as ANI-1x, ANI-2, or Behler-Parrinello Neural Network, among others), a convolutional neural network (such as SchNet, a crystal graph convolution neural network, or a tensor field network, among others), a graph neural network (such as DimeNet, PhysNet, or neural message passing for quantum chemistry, among others) recurrent neural networks (such as DeepMD, Long Short-Term Memory Networks in Molecular dynamics, or GRU-based potential models, among others), attention mechanisms and transformers (such as molecular transformers, attentive fingerprint, or Chemformer, among others), and/or message passing neural networks (such as DimeNet++, graph attention networks for molecular modeling, or MEGNet, among others).

Indeed, as illustrated, the implicit delta learning system 100 can cause the backbone neural network 306 to generate a feature representation 308. Indeed, the implicit delta learning system 100 can utilize the backbone neural network 306 to generate the feature representation 308 of the training compound geometry 302. As used herein, the term “feature representation” refers to a structured encoding or feature vector generated by a machine learning model. For example, a feature representation can include a latent/hidden feature vector generated by a backbone neural network from a compound. The feature representation can be a latent shared representation of a training compound geometry and/or a compound geometry that the prediction heads (e.g., the high fidelity prediction heads, the low fidelity prediction heads, and any other prediction heads such as medium fidelity prediction heads) utilize to generate quantum mechanics property predictions.

As illustrated in FIG. 3A, the implicit delta learning system 100 provides the feature representation 308 to a first prediction head 312 (corresponding to a first quantum mechanics representation class 310) and a second prediction head 318 (corresponding to a second quantum mechanics representation class 316). The first quantum mechanics representation class 310 can include high fidelity ab initio quantum mechanics property prediction having a high level of accuracy (e.g., compared to the second quantum mechanics representation class 316). Similarly, the second quantum mechanics representation class 316 can include low fidelity ab initio quantum mechanics property predictions having a low level of accuracy (e.g., compared to the first quantum mechanics representation class 310).

Moreover, as shown in FIG. 3A, the implicit delta learning system 100 can cause the first prediction head 312 to generate a first quantum mechanics property prediction 314 of the feature representation 308. Additionally, the implicit delta learning system 100 can cause the second prediction head 318 to generate a second quantum mechanics property prediction 320 of the feature representation 308. In other words, the implicit delta learning system 100 can generate two quantum mechanics property predictions (e.g., the first quantum mechanics property prediction 314 and the second quantum mechanics property prediction 320) corresponding to different classes (e.g., the first quantum mechanics representation class 310 and the second quantum mechanics representation class 316) from the feature representation 308.

As illustrated, the implicit delta learning system 100 can utilize a first quantum mechanics model 322 to generate a first ground truth 324 for the training compound geometry 302. The implicit delta learning system 100 can determine which model to utilize for the first quantum mechanics model 322 according to the first quantum mechanics representation class 310. Accordingly, the implicit delta learning system 100 can cause the first quantum mechanics model 322 to generate the first ground truth 324 so that it aligns to the same class (e.g., the first quantum mechanics representation class 310) as the first prediction head 312. In some implementations, the implicit delta learning system 100 access the first ground truth 324 (e.g., from a third-party or server, in the case where the first ground truth 324 is pre-generated).

Additionally, the implicit delta learning system 100 can utilize a second quantum mechanics model 328 to generate a second ground truth 330 for the training compound geometry 302. The implicit delta learning system 100 can determine which model to use for the second quantum mechanics model 328 according to the second quantum mechanics representation class 316. Accordingly, the implicit delta learning system 100 can cause the second quantum mechanics model 328 to generate the second ground truth 330 so that it corresponds to the same class (e.g., the second quantum mechanics representation class 316) as the second prediction head 318.

Further, as shown, the implicit delta learning system 100 can perform an act 338 to compare the first quantum mechanics property prediction 314 with the first ground truth 324 to determine a first measure of loss. The implicit delta learning system 100 can utilize the first measure of loss to perform an act 336 to update parameters of the first prediction head 312. Additionally, the implicit delta learning system 100 can utilize the first measure of loss to update parameters of the backbone neural network 306.

Moreover, as illustrated, the implicit delta learning system 100 can perform an act 340 to compare the second quantum mechanics property prediction 320 with the second ground truth 330 to determine a second measure of loss. The implicit delta learning system 100 can utilize the second measure of loss to perform an act 332 to update parameters of the second prediction head 318. Additionally, the implicit delta learning system 100 can utilize the second measure of loss to update parameters of the backbone neural network 306.

Thus, as shown in FIG. 3A, the implicit delta learning system 100 can utilize multiple ground truths from multiple different quantum mechanics representation classes for the same training compound geometry (and the same feature representation). Indeed, for a single training compound geometry, the implicit delta learning system 100 can generate a feature representation and then utilize multiple prediction heads to generate multiple predictions. By comparing these predictions with different quantum mechanics property predictions from different quantum mechanics representation classes, the implicit delta learning system 100 can train the backbone neural network 306 and the individual prediction heads corresponding to the different quantum mechanics representation classes.

Although FIG. 3A illustrates utilizing multiple ground truth quantum mechanics property predictions for a single training compound geometry, the implicit delta learning system 100 can also utilize different ground truth quantum mechanics property predictions from different classes for differing training compound geometries. Indeed, the implicit delta learning system 100 can train a neural network potential model utilizing a first sample that only has a high-fidelity ground truth, and a second sample that only has a low-fidelity ground truth. For example, FIG. 3B illustrates the implicit delta learning system 100 generating multiple feature representations from multiple training compound geometries and utilizing the multiple feature representations to generate multiple quantum mechanics property predictions.

As illustrated in FIG. 3B, the implicit delta learning system 100 can provide a first training compound geometry 352 and a second training compound geometry 354 to a neural network potential model 392 (“NNP 392”). For example, the NNP 392 can include the NNP 304 of FIG. 3A. The implicit delta learning system 100 can select the first training compound geometry 352 and the second training compound geometry 354 from among a plurality of training compound geometry. Moreover, the implicit delta learning system 100 can cause a backbone neural network 356 (e.g., the backbone neural network 306 of FIG. 3A) of the NNP 392 to generate a first feature representation 362 of the first training compound geometry 352. Additionally, the implicit delta learning system 100 can cause the backbone neural network 356 to generate a second feature representation 365 of the second training compound geometry 354.

Moreover, as shown, the implicit delta learning system 100 can cause a first prediction head 364 corresponding to a first quantum mechanics representation class 360 to generate a first quantum mechanics property prediction 366 of the first feature representation 362. Additionally, the implicit delta learning system 100 can cause a second prediction head 372 corresponding to a second quantum mechanics representation class 370 to generate a second quantum mechanics property prediction 374 of the second feature representation 365.

As illustrated, the implicit delta learning system 100 can cause a first quantum mechanics model 376 to generate (or receive/access) a first ground truth 378. Specifically, the implicit delta learning system 100 can determine the first quantum mechanics model 376 according to the first quantum mechanics representation class 360. Moreover, the implicit delta learning system 100 can cause the first quantum mechanics model 376 to generate the first ground truth 378 according to the first quantum mechanics representation class 360 corresponding to the first prediction head 364.

Moreover, as shown, the implicit delta learning system 100 can perform an act 388 to compare the first quantum mechanics property prediction 366 with the first ground truth 378 to determine a first measure of loss. The implicit delta learning system 100 can utilize the first measure of loss to perform an act 380 to update parameters of the first prediction head 364. Moreover, the implicit delta learning system 100 can utilize the first measure of loss to update parameters of the backbone neural network 356.

Additionally, as illustrated, the implicit delta learning system 100 can cause a second quantum mechanics model 382 to generate (or access/receive) a second ground truth 384. Indeed, the implicit delta learning system 100 can determine the second quantum mechanics model 382 according to the second quantum mechanics representation class 370. Moreover, the implicit delta learning system 100 can cause the second quantum mechanics model 382 to generate the second ground truth 384 according to the second quantum mechanics representation class 370.

Indeed, as shown, the implicit delta learning system 100 can perform an act 390 to compare the second quantum mechanics property prediction 374 with the second ground truth 384 to determine a second measure of loss. The implicit delta learning system 100 can utilize the second measure of loss to perform an act 386 to update parameters of the second prediction head 372. Moreover, the implicit delta learning system 100 can utilize the second measure of loss to update parameters of the backbone neural network 356.

Thus, as shown in FIG. 3B, the implicit delta learning system 100 can utilize different training compound geometries that have different ground truths corresponding to different quantum mechanics representation classes. Despite having different ground truths corresponding to different classes, each sample can improve the accuracy of the backbone neural network 356 and the individual prediction heads corresponding to each particular sample.

In one or more implementations the implicit delta learning system 100 can train NNPs by minimizing an energy-matching mean squared error (MSE) loss function. Specifically, the implicit delta learning system 100 can train high fidelity NNPs utilizing the following equation:

$$ℒ = \frac{1}{N} {\sum }_{i = 1}^{N} {[ {E}_{i}^{H F} - {\hat{E}}_{θ}^{H F} ( {S}_{i} ) ]}^{2}$$

where Si is a conformation of a molecule with Ki atoms, typically represented by positions Rᵢ ∈ R^{Ki x 3} and atomic numbers Zᵢ ∈ N^{Ki}, Eᵢ^{HF} is the target energy, and Ê_{θ}^{HF} (Sᵢ) is the predicted energy of the NNP parameterized by θ.

Instead of directly learning and predicting the HF energy Ê_{θ}^{HF}(Sᵢ), some conventional methods, such as delta learning, learn to predict the energy difference with respect to a low frequency energy E^{LF}. Thereafter, during inference, for conventional systems to predict the high frequency energy requires the conventional system to compute the low frequency energy and then the delta, i.e., Ê_{θ}^{HF}(Sᵢ)=Eᵢ^{LF}+ΔÊ_{θ}^{HF-LF} (Sᵢ). Conventional systems learn the parameters of the delta model by minimizing the MSE loss function using samples including both the high frequency and low frequency labels as

$${ℒ}^{Δ} = \frac{1}{N} {\sum }_{i = 1}^{N}$$

[(Eᵢ^{HF}−Eᵢ^{LF})−ΔE_{θ}^{NN}(Sᵢ)]². Accordingly, due to the form of loss, traditional methods require pairs of high fidelity and low fidelity energies for any given geometry in training data, which makes data collection for conventional systems computationally expensive. Moreover, delta-learning methods can be straightforwardly applied to data sets with one low fidelity and one high fidelity method, however, conventional systems lack the operational flexibility to generalize delta-learning methods to multiple low fidelity methods and a high fidelity method.

Conversely, the implicit delta learning system 100 can generalize easily to multiple low-fidelity methods and does not need low fidelity calculations to be accurate during inference. Indeed, the implicit delta learning system 100 leverages low fidelity and high fidelity data simultaneously (e.g., compared to the traditional two stages of pre-training and fine-tuning implemented by traditional systems)

Indeed, in one or more implementations the implicit delta learning system 100 is trained utilizing the following equation:

$${ℒ}^{MT} = \frac{1}{M} {\sum }_{i = 1}^{N} {\sum }_{h = 1}^{H} {I}_{i , h} \times {[ {E}_{i , h} - {\hat{E}}_{θ , h} ( {S}_{i} ) ]}^{2}$$

where M is the number of energy labels in the dataset, N is the number of geometries, H is the number of quantum mechanics methods (both high fidelity and low fidelity) considered, I_{i,h} is 0 if the label E_{i,h} is missing, otherwise 1, and Ê_{θ,h} (Sᵢ) is the energy prediction of head h for the geometry Sᵢ.

The implicit delta learning system 100 can implement the foregoing methodologies to leverage the feature representations created by the backbone neural network. Indeed, experimental implementations of the implicit delta learning system utilize a backbone neural network to generate a latent feature representation, thereby allowing prediction heads (e.g., high fidelity prediction heads and low fidelity prediction heads) to decode their respective energy values from the latent feature representation (e.g., a shared representation of the input geometry). Accordingly, experimental implementations of the implicit delta learning system leverage multiple low fidelity labels to improve high fidelity prediction accuracy.

Moreover, as discussed above with respect to FIG. 1, the implicit delta learning system 100 can train a plurality of prediction heads within each quantum mechanics representation class. Specifically, the implicit delta learning system 100 can train each prediction head of a first plurality of prediction heads to generate a quantum mechanics property prediction at an accuracy level corresponding to a first quantum mechanics representation class (e.g., each prediction head of the first plurality of prediction heads generates a quantum mechanics property prediction for a different quantum mechanics property). Similarly, the implicit delta learning system 100 can teach each prediction head of a second plurality of prediction heads to generate a quantum mechanics property prediction at an accuracy level corresponding to a second quantum mechanics representation class.

Moreover, when training a neural network potential model, the implicit delta learning system 100 can determine to mix, leverage, and/or otherwise combine the training methodologies described above with respect to FIGS. 3A-3B. That is to say, the implicit delta learning system 100 can utilize a duality of training methods when training the NNP. The implicit delta learning system 100 can utilize a first training method by generating a feature representation from a training compound geometry. Further, as part of the first training method, the implicit delta learning system 100 can provide the feature representation to a first plurality of prediction heads of a first quantum mechanics representation class. The implicit delta learning system 100 can, as part of the first training method, provide the feature representation to a second plurality of prediction heads of a second quantum mechanics representation class.

Additionally, the implicit delta learning system 100 can utilize a second training method. The implicit delta learning system 100 can implement the second training method by generating a first feature representation from a first training compound geometry and generating a second feature representation from a second training compound geometry. Moreover, as part of the second training method, the implicit delta learning system 100 can provide the first feature representation to the first plurality of prediction heads of the first quantum mechanics representation class. Further, the implicit delta learning system 100 can provide the second feature representation to the second plurality of prediction heads of the second quantum mechanics representation class.

The implicit delta learning system 100 can determine to implement the first training method, the second training method, or both training methods in parallel or in series. By leveraging the duality of training methods, the implicit delta learning system 100 can increase the accuracy, efficiency, and operational flexibility of implementing systems.

Additionally, while FIGS. 3A-3B illustrate the implicit delta learning system 100 determining multiple measures of loss (e.g., such as one measure of loss per quantum mechanics representation class), in some embodiments, the implicit delta learning system 100 can determine a total measure of loss and utilize the total measure of loss to update parameters of the backbone neural network and/or the prediction heads. For example, the implicit delta learning system 100 can determine the total measure of loss by combining measures of loss from respective quantum mechanics representation classes and/or multiple quantum mechanics property predictions within a quantum mechanics representation class. The implicit delta learning system 100 can utilize the total measure of loss to update parameters of the neural network potential model. For example, the implicit delta learning system 100 can backpropagate the total measure of loss to the backbone neural network and or to prediction heads (e.g., a first prediction head of a first quantum mechanics representation class and/or a second prediction head of a second quantum mechanics representation class).

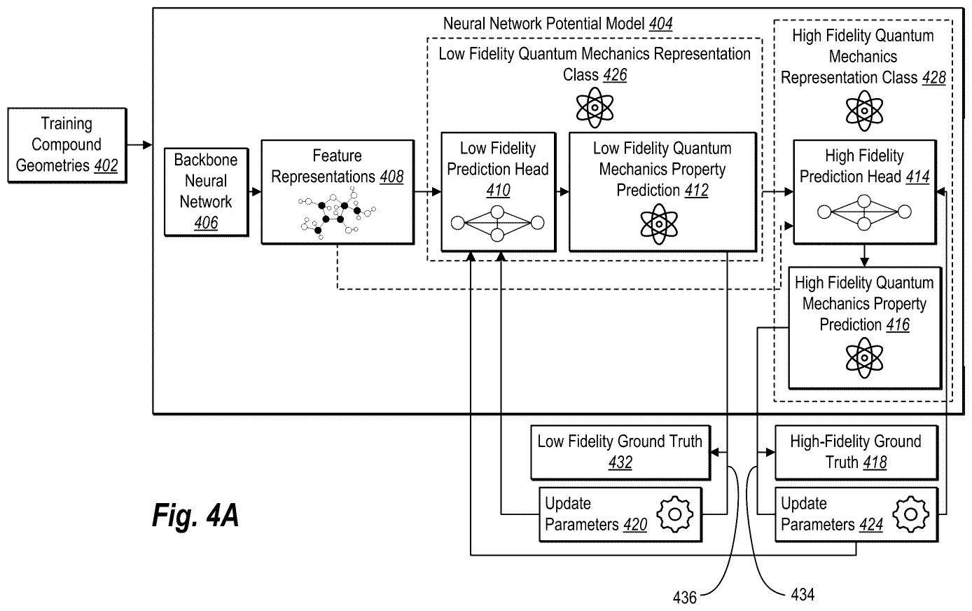

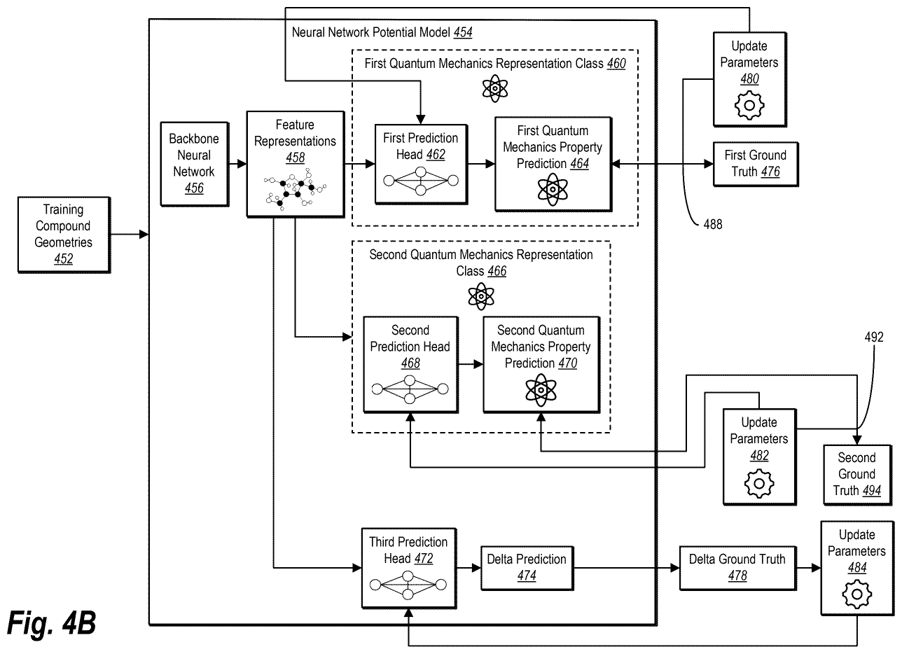

As previously mentioned, the implicit delta learning system 100 can utilize alternative neural network potential model architectures to generate quantum mechanics property predictions of training compound geometries. FIG. 4A illustrates the implicit delta learning system 100 utilizing an additional architecture to generate feature representations from training compound geometries, generate a low fidelity quantum mechanics property prediction from the feature representation, and generate a high fidelity quantum mechanics property prediction from the low fidelity property prediction.

As illustrated in FIG. 4A, the implicit delta learning system 100 can input training compound geometries 402 into a neural network potential model 404 (“NNP 404”). In some embodiments, the implicit delta learning system 100 can determine how many of the training compound geometries 402 to input into the NNP 404 (e.g., in some embodiments, the implicit delta learning system 100 can input one training compound geometry into the NNP 404, in some embodiments, the implicit delta learning system 100 can input multiple training compound geometries into the NNP 404).

The implicit delta learning system 100 utilizes a backbone neural network 406 of the NNP 404 to generate feature representations 408 from the training compound geometries 402. In particular, the implicit delta learning system 100 generates a feature representation for each training compound geometry.

As illustrated, the implicit delta learning system 100 analyzes the feature representations 408 utilizing a low fidelity prediction head 410 corresponding to a low fidelity quantum mechanics representation class 426 (e.g., a prediction head labeled for training to generate low fidelity property predictions). The implicit delta learning system 100 can cause the low fidelity prediction head 410 to generate a low fidelity quantum mechanics property prediction 412. The implicit delta learning system 100 can perform an act 436 to compare the low fidelity quantum mechanics property prediction 412 with a low fidelity ground truth 432 corresponding to the low fidelity quantum mechanics representation class 426 to determine a second measure of loss. Thereafter, the implicit delta learning system 100 can utilize the second measure of loss to perform an act 420 to update parameters of the low fidelity prediction head 410. Additionally, the implicit delta learning system 100 can utilize the second measure of loss to update parameters of the backbone neural network 406.

As denoted by the dotted lines of the act 420, the low fidelity ground truth 432, and the act 436, the implicit delta learning system 100 can optionally determine to perform the act 436 to compare the low fidelity quantum mechanics property prediction 412 with the low fidelity ground truth 432. Moreover, responsive to performing the act 436, the implicit delta learning system 100 can optionally determine to perform the act 420 to update the parameters of the low fidelity prediction head 410.

As shown, the implicit delta learning system 100 can analyze the low fidelity quantum mechanics property prediction 412 utilizing a high fidelity prediction head 414 corresponding to a high fidelity quantum mechanics representation class 428 (e.g., a prediction head labeled for training to generate low high fidelity property predictions). The implicit delta learning system 100 can cause the high fidelity prediction head 414 to generate a high fidelity quantum mechanics property prediction 416. Explained differently, the implicit delta learning system 100 can utilize a high fidelity prediction head (e.g., the high fidelity prediction head 414) to generate a high fidelity quantum mechanics property prediction (e.g., the high fidelity quantum mechanics property prediction 416) from a low fidelity quantum mechanics property prediction (e.g., the low fidelity quantum mechanics property prediction 412).

Responsive to generating the high fidelity quantum mechanics property prediction 416, the implicit delta learning system 100 can perform an act 434 to compare the high fidelity quantum mechanics property prediction 416 with a high fidelity ground truth 418 corresponding to the high fidelity quantum mechanics representation class 428 to determine a first measure of loss. The implicit delta learning system 100 can utilize the first measure of loss to perform an act 424 to update parameters of the high fidelity prediction head 414 and/or the low fidelity prediction head 410. Additionally, the implicit delta learning system 100 can utilize the second measure of loss to update parameters of the backbone neural network 406.

As previously mentioned, the implicit delta learning system 100 can implement alternative architectures to generate quantum mechanics property predictions of different classes. FIG. 4B illustrates the implicit delta learning system 100 generating a first quantum mechanics property prediction, a second quantum mechanics property prediction, and a delta prediction for a difference in quality between the first quantum mechanics property prediction and the second quantum mechanics property prediction.

As illustrated in FIG. 4B, the implicit delta learning system 100 and training compound geometries 452 into a neural network potential model 454 (“NNP 454”). The implicit delta learning system 100 can cause a backbone neural network 456 of the NNP 454 to generate feature representations 458 from the training compound geometries.

As shown, the implicit delta learning system 100 can analyze the feature representations 458 utilizing a first prediction head 462 corresponding to a first quantum mechanics representation class 460. The implicit delta learning system 100 can cause the first prediction head 462 to generate a first quantum mechanics property prediction 464 (e.g., a high fidelity quantum mechanics property prediction).

As illustrated, the implicit delta learning system 100 can perform an act 488 to compare the first quantum mechanics property prediction 464 with a first ground truth of the first quantum mechanics representation class 460 to determine a first measure of loss. Responsive to determining the first measure of loss, the implicit delta learning system 100 can perform an act 480 to update parameters of the first prediction head 462. Additionally, the implicit delta learning system 100 can utilize the first measure of loss to update parameters of the backbone neural network 456.

Moreover, as shown, the implicit delta learning system 100 can analyze the feature representations 458 utilizing a second prediction head 468 of a second quantum mechanics representation class 466 (e.g., a prediction head that utilizes low fidelity methods to generate quantum mechanics property predictions). The implicit delta learning system 100 can cause the second prediction head 468 to generate a second quantum mechanics property prediction 470 (e.g., a low fidelity quantum mechanics property prediction). Responsive to generating the second quantum mechanics property prediction 470, the implicit delta learning system 100 can perform an act 492 to compare the second quantum mechanics property prediction 470 with a second ground truth 494 of the second quantum mechanics representation class 466 to determine a second measure of loss. The implicit delta learning system 100 can utilize the second measure of loss to perform an act 482 to update parameters of the second prediction head 468. Additionally, the implicit delta learning system 100 can utilize the second measure of loss to update parameters of the backbone neural network 456.

As illustrated, the implicit delta learning system 100 can analyze the feature representations 458 utilizing a third prediction head 472. The implicit delta learning system 100 can cause the third prediction head 472 to generate a delta prediction 474 (e.g., a prediction in a difference in accuracy between the first quantum mechanics property prediction 464 and the second quantum mechanics property prediction 470). Additionally, the implicit delta learning system 100 can cause the implicit delta learning system 100 do determine a delta ground truth 478 by determining a difference between the first ground truth 476 and the second ground truth 494 (e.g., the delta ground truth 478 can be a function of the first ground truth 476 and the second ground truth 494).

Based on determining the delta ground truth 478, the implicit delta learning system 100 can compare the delta prediction 474 to the delta ground truth 478 to determine a third measure of loss. Moreover, in some embodiments, the implicit delta learning system 100 can determine the delta ground truth 478 by determining a difference in two property predictions. For example, the implicit delta learning system 100 can determine the delta ground truth 478 by determining the difference between the second quantum mechanics property prediction 470 and the second ground truth 494 (e.g., subtracting the second ground truth 494 from the second quantum mechanics property prediction). In some implementations, the implicit delta learning system 100 can determine the delta ground truth 478 by determining the difference between the first ground truth 476 and the second ground truth 494 or the difference between the first quantum mechanics property prediction 464 and the second quantum mechanics property prediction 470. Responsive to determining the third measure of loss, the implicit delta learning system 100 can perform an act 484 to update parameters of the third prediction head 472. Additionally, the implicit delta learning system 100 can utilize the third measure of loss to update parameters of the backbone neural network 456.

Moreover, in some embodiments, the implicit delta learning system 100 can determine to generate a quantum mechanics property prediction having a threshold level of accuracy. Indeed, the implicit delta learning system 100 can intelligently determine how many high fidelity quantum mechanics property predictions and how many low fidelity quantum mechanics property predictions to sample in order to generate the quantum mechanics property prediction having the threshold level of accuracy. For example, the implicit delta learning system 100 can determine to generate a first quantum mechanics property prediction having a first threshold level of accuracy. Responsive to this determination, the implicit delta learning system 100 can determine to sample a first number of high fidelity quantum mechanics property predictions and a second number of low fidelity quantum mechanics property predictions in order to generate the first quantum mechanics property prediction having the first threshold level of accuracy.

Moreover, as discussed previously with regard to FIG. 1, the implicit delta learning system 100 can train a first plurality of low fidelity prediction heads such that each of the first plurality of low fidelity prediction heads generates a different low fidelity quantum mechanics property prediction. Additionally, the implicit delta learning system 100 can train a second plurality of high fidelity prediction heads such that each of the second plurality of high fidelity prediction heads generates a different high fidelity quantum mechanics property prediction.

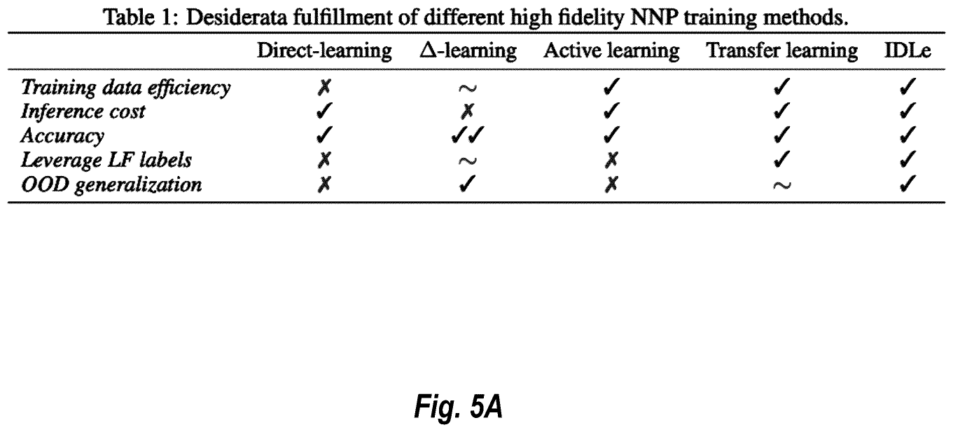

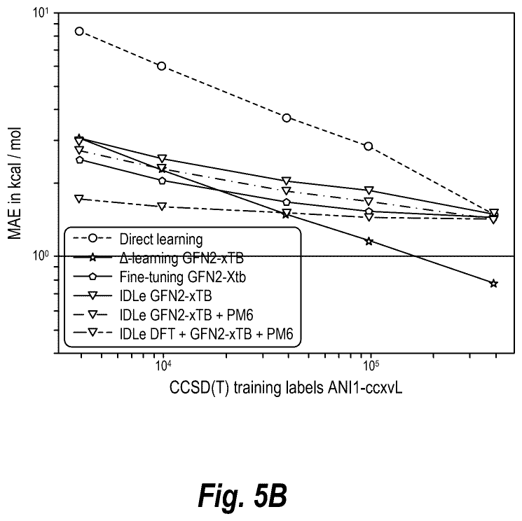

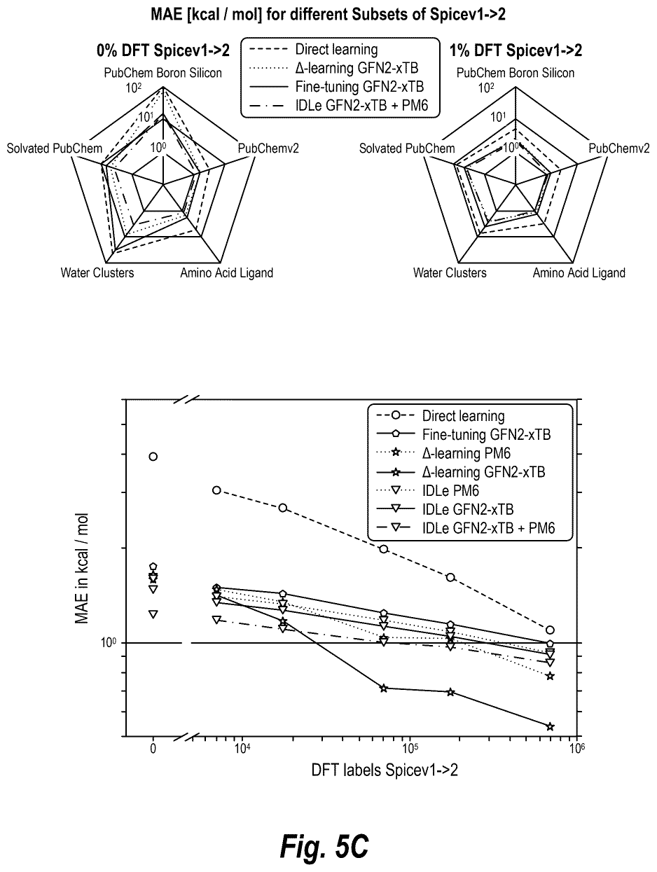

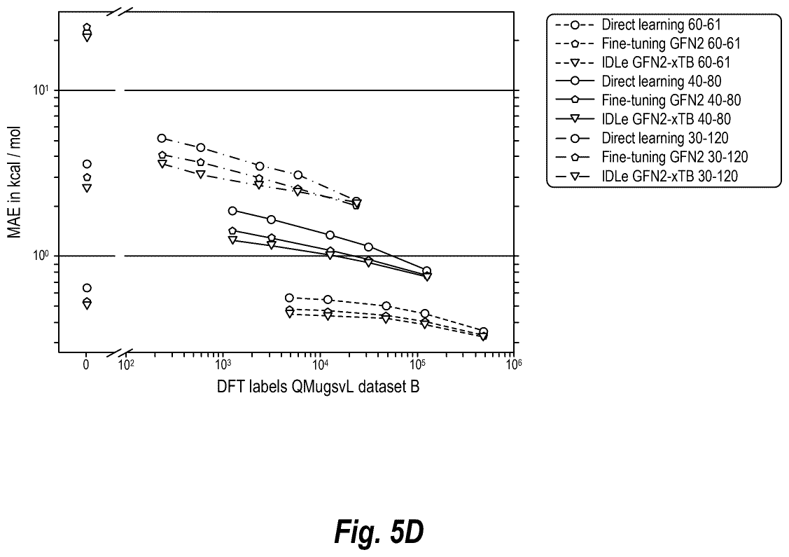

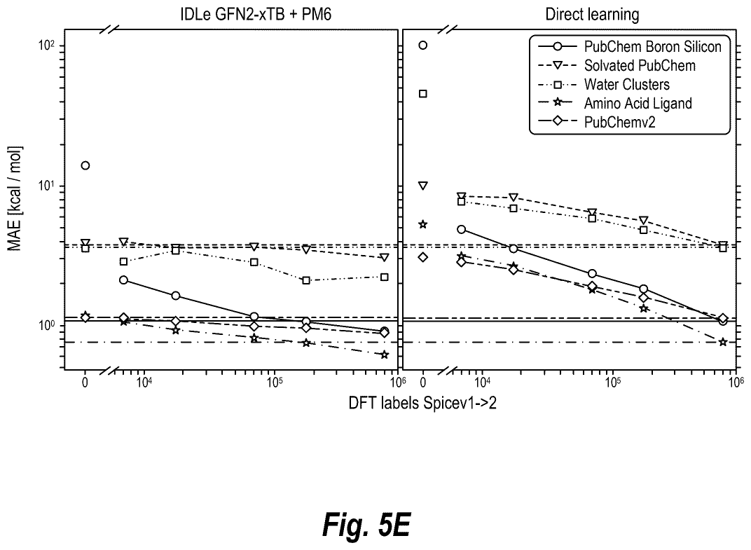

As previously discussed, the implicit delta learning system improves the functionality of conventional systems. FIGS. 5A-5E illustrate several advantages and/or results achieved by an experimental implementation of the implicit delta learning system compared to conventional systems. In FIGS. 5A-5E, experimental implementations of the implicit delta learning system are labeled as “IDLe.”

FIG. 5A illustrates the advantages of an experimental implementation of the implicit delta learning system compared to conventional systems. Specifically, FIG. 5A compares the performances of direct-learning systems, delta learning systems, active learning systems, transfer learning systems, and the experimental implementation of the implicit delta learning system in the areas of training data efficiency, inference cost, accuracy, leveraging of low fidelity labels, and out-of-distribution generalization. As illustrated, the experimental implementation of the implicit delta learning system achieves a more complete, holistic performance than conventional systems. For example, as previously discussed, the implicit delta learning system is more generalizable to multiple low fidelity methods working in tandem with a high fidelity method. In addition, the implicit delta learning system increases the training data efficiency compared to conventional systems by leveraging low fidelity and high fidelity methods. Moreover, as previously discussed, the implicit delta learning system decreases the computational expenses associated with inference.

For FIGS. 5B-5E, the experimental implementation of the implicit delta learning system 100 performed calculations utilizing the following equation:

$${\overline{E}}_{i , j}^{QM} = \frac{1}{{σ}_{j}} ( {E}_{i . j}^{QM} - {\sum }_{k = 1}^{{K}_{1}} {μ}_{j , k} )$$

where Ē is the computed normalized energy levels, j is the level of QM theory, μ_{j,k} is a linear model, and σⱼ is the mean residual energy per atom.

For all experiments, the experimental implementation of the implicit delta learning system 100 performed an 80%-10%-10% training-validation-test split. Further, in all experiments, the implicit delta learning system varied the amount of high fidelity labels in the training and validation set from 1%, 2.5%, 10%, 25%, and 100%.

FIG. 5B illustrates a performance of experimental implementations of the implicit delta learning system in an IID setting utilizing coupled cluster CCSD (T) as HF labels. Specifically, FIG. 5B illustrates the MAE of the experimental implementations of the implicit delta learning system compared to conventional systems. Indeed, the experimental implementations of the implicit delta learning system consistently outperform conventional systems that utilize direct learning and fine-tuning methods. Additionally, the experimental implementations of the implicit delta learning system outperform conventional systems that utilize delta learning methods at smaller magnitudes of CCSD (T) training labels.

As illustrated in FIG. 5B, the experimental implementations of the implicit delta learning system 100 achieve increased performances by leveraging LF labels. Moreover, a first experimental implementation of the implicit delta learning system utilizing Parameterized Method 6 (PM6) and Geometry, Frequency, Noncovalent, extended Tight Binding 2 (GFN2-xTB) achieves increased performance (e.g., decreased the MAE) compared to a second experimental implementation of the implicit delta learning system utilizing GFN2-xTB alone.

FIG. 5C illustrates the MAE of experimental implementations of the implicit delta learning system when utilizing out-of-distribution (OOD) data sets. Specifically, experimental implementations of the implicit delta learning system utilize SpiceV1 and Spice1->2 (e.g., the difference between SpiceV2 and SpiceV1), excluding the PubChem-Boron-Silicon subset of SpiceV1->2, as training data sets.

As illustrated, a first experimental implementation of the implicit delta learning system utilizing Tight Binding (TB) methodologies outperforms delta learning and fine-tuning methodologies. Additionally, a second experimental implementation of the implicit delta learning system utilizing TB and Hartree-Fock-based Semi Empirical (SE) methodologies achieves the same performance as a direct learning baseline that utilizes approximately 50% HF labels. A third experimental implementation of the implicit delta learning system utilizing 2.5% HF labels and TB+SF labels achieves the same accuracy as 100% of Spicev1->2. Indeed, as illustrated by FIG. 5C, experimental implementations of the implicit delta learning system exhibit increased chemical transferability.

FIG. 5D illustrates the results (e.g., MAE) of experimental embodiments of the implicit delta learning system and conventional systems extrapolating to larger molecules (e.g., compared to the average molecule size in training data sets. Specifically, experimenters split QMugsvL into training data sets. Each training data set included training dataset A of molecules having up to na atoms, and an OOD dataset B of molecules having at least ng atoms. To simulate increasing levels of OOD difficulty, experimenters performed three splits (e.g., created three training data sets, each training data set including a training data set A and a training data set B.

As shown in FIG. 5D, for all splits, experimental implementations of the implicit delta learning system utilizing GFN2-xTB outperform direct learning and fine tuning methods on data sets B. Indeed, FIG. 5D demonstrates that the experimental implementations of the implicit delta learning system leverage HF fidelity the most efficiently, reaching almost the same performance as direct learning methodologies utilizing 25% DFT labels while the experimental embodiments of the implicit delta learning system utilize only 1% DFT labels.

FIG. 5E illustrates the data efficiency (e.g., MAE) of an experimental implementation of the implicit delta learning system utilizing GFN2-xTB and PM6 methodologies compared to a direct learning baseline for subsets of the Spicev1->2 data set. The experimental implementation of the implicit delta learning system achieves the same level of accuracy on the PubChemv2 and Water Cluster subsets as direct learning methodologies utilizing 100% HF data. Moreover, the error of the experimental implementation of the implicit delta learning system on the Solvated PubChem subset is only marginally larger than the direct learning baseline.

As shown in FIG. 5E, for subsets of Spicev1->2 with larger distribution shifts, the experimental embodiment of the implicit delta learning system require approximately 25% HF data to achieve the baseline of direct learning utilizing 100% HF data. Indeed, the experimental embodiment of the implicit delta learning system shows that when generating new data sets, utilizing SF methodologies to label conformers with small to medium distribution shifts achieves the same level of accuracy as the direct learning baseline, thereby increasing and improving the computational efficiency of the experimental implementation of the implicit delta learning system.

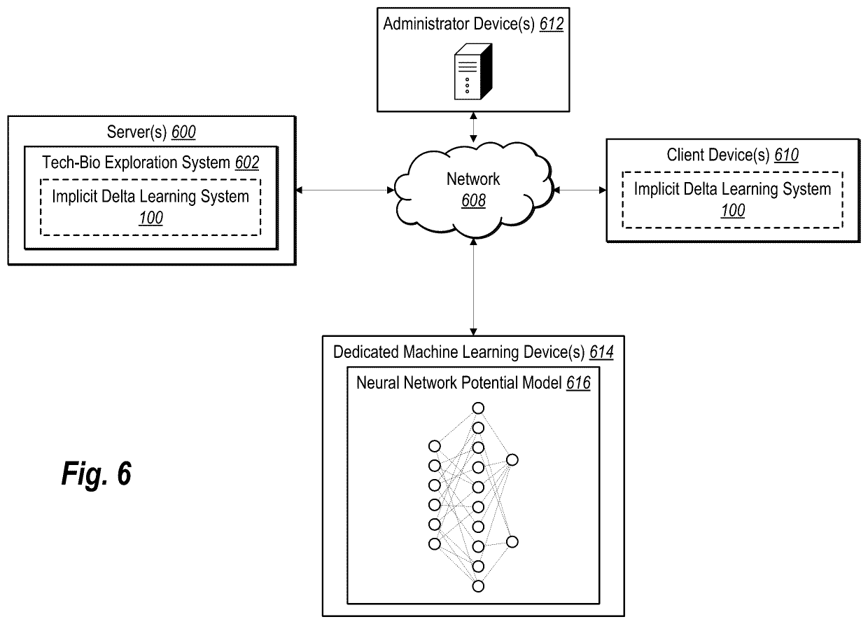

Additional detail regarding the implicit delta learning system 100 environment will now be provided with reference to FIG. 6. In particular, FIG. 6 illustrates a schematic diagram of a system environment in which the implicit delta learning system 100 can operate in accordance with one or more embodiments.

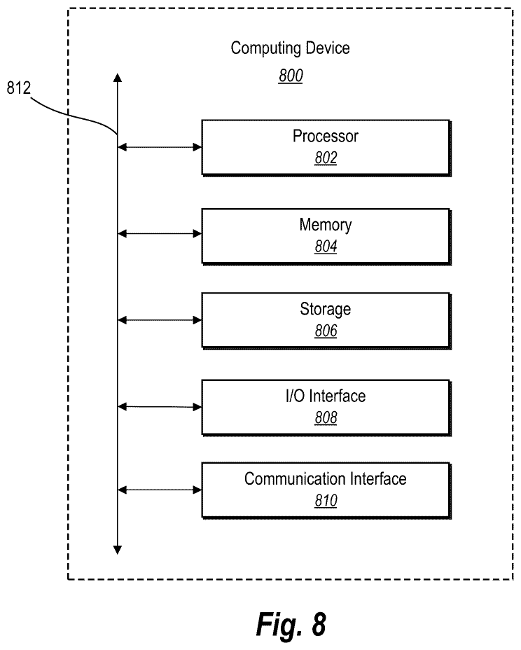

As shown in FIG. 6, the environment includes server(s) 600 (which includes a tech-bio exploration system 602 and the implicit delta learning system 100), dedicated machine learning device(s) 614, a network 608, client device(s) 610 and administrator device(s) 612. As further illustrated in FIG. 6, the various computing devices within the environment can communicate via the network 608. Although FIG. 6 illustrates the implicit delta learning system 100 being implemented by a particular component and/or device within the environment, the implicit delta learning system 100 can be implemented, in whole or in part, by other computing devices and/or components in the environment (e.g., the additional device(s)). Additional description regarding the illustrated computing devices is provided with respect to FIG. 8 below.

As shown in FIG. 6, the server(s) 600 (e.g., one or more local servers operated by a particular entity) can include the tech-bio exploration system 602. In some embodiments, the tech-bio exploration system 602 can determine, store, generate, and/or display tech-bio information including maps of biology, experiments from various sources, and/or machine learning tech-bio predictions. For instance, the tech-bio exploration system 602 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal). Moreover, the tech-bio exploration system 602 provides an environment for operating, executing, and managing complex drug discovery pipelines.

For instance, the tech-bio exploration system 602 can generate and access experimental results corresponding to gene sequences, protein shapes/folding, protein/compound interactions, phenotypes resulting from various interventions or perturbations (e.g., gene knockout sequences or compound treatments), and/or invivo experimentation on various treatments in living animals. By analyzing these signals (e.g., utilizing various machine learning models), the tech-bio exploration system 602 can generate or determine a variety of predictions and inter-relationships for improving treatments/interventions.

To illustrate, the tech-bio exploration system 602 can generate maps of biology indicating biological inter-relationships or similarities between these various input signals to discover potential new treatments as part of the complex compound discovery process. For example, the tech-bio exploration system 602 can utilize machine learning and/or maps of biology to identify a similarity between a first gene associated with disease treatment and a second gene previously unassociated with the disease based on a similarity in resulting phenotypes from gene knockout experiments. The tech-bio exploration system 602 can then identify new treatments based on the gene similarity (e.g., by targeting compounds the impact the second gene). Similarly, the tech-bio exploration system 602 can analyze signals from a variety of sources (e.g., protein interactions, or invivo experiments) to predict efficacious treatments based on various levels of biological data.

The tech-bio exploration system 602 can generate GUIs comprising dynamic user interface elements to convey tech-bio information and receive user input for intelligently exploring tech-bio information. Indeed, as mentioned above, the tech-bio exploration system 602 can generate GUIs displaying different maps of biology that intuitively and efficiently express complex interactions between different biological systems for identifying improved treatment solutions. Furthermore, the tech-bio exploration system 602 can also electronically communicate tech-bio information between various computing devices.

As shown in FIG. 6, the tech-bio exploration system 602 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 602 collects, manages, and transmits data across a variety of different entities, accounts, and devices. In some cases, the tech-bio exploration system 602 is a network system that facilitates access to (and analysis of) tech-bio information within a centralized operating system. Indeed, the tech-bio exploration system 602 can link data from different network-based research institutions to generate and analyze maps of biology.

As shown in FIG. 6, the tech-bio exploration system 602 can include a system that comprises the implicit delta learning system 100 that generates, stores, manages, transmits data pertaining to the generation of feature representations from query compound geometries and/or training compound geometries. The implicit delta learning system 100 can subsequently generate multiple classes of quantum mechanics property predictions from the feature representations. For example, in context of the above description for the tech-bio exploration system 602, in some embodiments the tech-bio exploration system 602 further utilizes the implicit delta learning system 100 to enhance the coordination between various groups involved in the drug discovery process. For instance, the implicit delta learning system 100 works in tandem with the tech-bio exploration system 602 to generate feature representations, generate multiple classes of quantum mechanics property predictions from the feature representations, and utilize the quantum mechanics property predictions to generate bioactivity predictions, transmit the bioactivity predictions to one or more devices, and initiate one or more downstream model predictions or processes.

As also illustrated in FIG. 6, the environment includes the client device(s) 610. As mentioned above, the client device(s) 610 can be involved in the process of drug discovery. Thus, for example, the client device(s) 610 can coordinate/manage a first stage of generating feature representations of query compound geometries and/or training compound geometries. Moreover, the client device(s) 610 can coordinate/manage a second stage such as generating quantum mechanics property predictions (e.g., a high fidelity quantum mechanics property prediction and a low fidelity quantum mechanics property prediction) from the feature representations. Further, the client device(s) 610 can coordinate/manage a third stage of utilizing the high fidelity quantum mechanics property prediction to generate a biological prediction to generate one or more additional predictions or initiate one or more programs (IPG or ICG).

To illustrate, the client device(s) 610 can include computing devices that implement or manage a compound program generation stage of a compound discovery process. Similarly, the client device(s) 610 can include computing devices that implement or manage a compound lead generation stage and the client device(s) 610 can include computing devices that implement or manage a compound/dose selection stage. For example, the implicit delta learning system 100 can receive one or more requests to utilize the dedicated machine learning device(s) 614 to generate a high fidelity quantum mechanics property prediction from training compound geometries and/or query compound geometries. For instance, the implicit delta learning system 100 can receive additional requests from the client device(s) 610 that include generating the biological activity predictions from the quantum mechanics property prediction.

In some embodiments, the environment also includes additional device(s). For example, the implicit delta learning system 100 can utilize the additional device(s) to further operate and manage the completion of complex drug discovery pipelines. For instance, the additional device(s) include experimental device(s) and analytical device(s). Further, in some instances, the additional device(s) also include the computing devices discussed below in FIG. 8.

Furthermore, in one or more implementations, the client device(s) 610 include a client application. The client application can include instructions that (upon execution) cause the client device(s) 610 to perform various actions. For example, a user of a user account can interact with the client application on the client device(s) 610 to execute experiments or other multi-faceted processes and to further access tech-bio information, initiate a request for a high fidelity quantum mechanics property prediction or a biological activity prediction. For instance, in some embodiments the implicit delta learning system 100 receives a request to generate a high fidelity quantum mechanics property prediction for a query compound geometry and/or a training compound geometry, and in response generates a high fidelity quantum mechanics property prediction and returns the high fidelity quantum mechanics property prediction to the client device(s) 610. In some instances, the transmittal of the high fidelity quantum mechanics property prediction to the client device(s) 610 causes the client device(s) 610 to execute an action (e.g., generate a downstream model prediction).

As shown, the environment can also include dedicated machine learning device(s) 614. For example, the dedicated machine learning device(s) 614 can include computing devices or virtual machines dedicated to training or implementing large-scale machine learning models. For example, the dedicated machine learning device(s) 614 can generate machine learning predictions and/or embeddings based on digital biological data (e.g., digital images of phenotypes resulting from different perturbations or compound-protein interactions from compound features). As shown, the dedicated machine learning device(s) 614 includes a neural network potential model 616. Thus, the implicit delta learning system 100 interacts with the dedicated machine learning device(s) 614 to generate quantum mechanics property predictions from training compound geometries and/or query compound geometries and generate biological activity predictions for the query compound geometries utilizing the high fidelity quantum mechanics property predictions.

The environment can also include experimental device(s). For example, the tech-bio exploration system 602 can interact with the experimental device(s) that include intelligent robotic devices and camera devices for generating and capturing digital images of cellular phenotypes resulting from different perturbations (e.g., genetic knockouts or compound treatments of stem cells). Similarly, the experimental device(s) can include camera devices and/or other sensors (e.g., heat or motion sensors) capturing real-time information from animals as part of invivo experimentation. The tech-bio exploration system 602 can also interact with a variety of other experimental device(s) such as devices for determining, generating, or extracting gene sequences or protein information. For example, the experimental device(s) may include computing devices linked to biosensorselectrophysiological platforms, x-ray crystallography machines, liquid chromatography mass spectrometry systems, nuclear magnetic resonance spectrometers, mass spectrometers. In some implementations, the implicit delta learning system 100 generates feature representations from training compound geometries and/or query compound geometries, generates multiple classes of quantum mechanics property predictions from the feature representations and further determines to employ or utilize one or more experimental devices (e.g., to initiate one or more experiments based on the high fidelity quantum mechanics property prediction).

As further shown in FIG. 6, the environment includes the network 608. As mentioned above, the network 608 can enable communication between components of the environment. In one or more embodiments, the network 608 may include a suitable network and may communicate using a various number of communication platforms and technologies suitable for transmitting data and/or communication signals, examples of which are described with reference to FIG. 8. Furthermore, although FIG. 6 illustrates computing devices communicating via the network 608, the various components of the environment can communicate and/or interact via other methods (e.g., communicate directly).

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

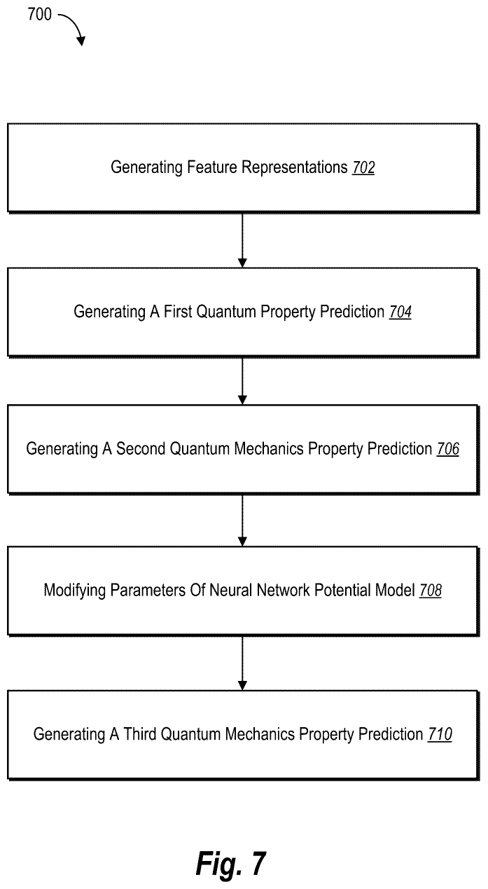

FIGS. 1-6, the corresponding text, and the examples provide a number of different systems, methods, and non-transitory computer readable media for generating feature representations for training compound geometries and/or compound geometries and utilizing the feature representation to generate quantum mechanics property predictions of various classes (e.g., high fidelity quantum mechanics property predictions and low fidelity quantum mechanics property predictions). In addition to the foregoing, embodiments can also be described in terms of flowcharts comprising acts for accomplishing a particular result. For example, FIG. 7 illustrates a flowchart of an example sequence of acts in accordance with one or more embodiments.

While FIG. 7 illustrates acts according to some embodiments, alternative embodiments may omit, add to, reorder, and/or modify any of the acts shown in FIG. 7. The acts of FIG. 7 can be performed as part of a method (e.g., a computer-implemented method). Alternatively, a non-transitory computer readable medium can comprise instructions, that when executed by one or more processors (e.g., at least one processor), cause a computing device to perform the acts of FIG. 7. In still further embodiments, the system can perform the acts of FIG. 7. Additionally, the acts described herein may be repeated or performed in parallel with one another or in parallel with different instances of the same or similar acts.

FIG. 7 illustrates an example series of acts 700 for generating quantum mechanics property predictions of different classes from a common feature representation. The series of acts 700 can include acts 702-710 of generating feature representations; generating a first quantum mechanics property prediction; generating a second quantum mechanics property prediction; modifying parameters of a neural network potential model; and generating a third quantum mechanics property prediction.

For example, in one or more embodiments, the acts 702-710 can include generating, utilizing a backbone neural network of a neural network potential model, feature representations from training compound geometries; generating, utilizing a first prediction head of the neural network potential model corresponding to a first quantum mechanics representation class, a first quantum mechanics property prediction from the feature representations; generating, utilizing a second prediction head of the neural network potential model corresponding to a second quantum mechanics representation class, a second quantum mechanics property prediction from the feature representations; modifying parameters of the neural network potential model by comparing the first quantum mechanics property prediction with a first ground truth from the first quantum mechanics representation class and the second quantum mechanics property prediction with a second ground truth from the second quantum mechanics representation class; and in response to receiving a query compound geometry, generating, utilizing the first prediction head of the neural network potential model, a third quantum mechanics property prediction from the query compound geometry.

In one or more implementations, the series of acts 700 can include comparing, utilizing a loss function, the first quantum mechanics property prediction with the first ground truth from the first quantum mechanics representation class to determine a first measure of loss; and modifying parameters of the backbone neural network and the first prediction head of the neural network potential model according to the first measure of loss.

Further, in some implementations, the series of acts 700 can include comparing, utilizing the loss function, the second quantum mechanics property prediction with the second ground truth from the second quantum mechanics representation class to determine a second measure of loss; and modifying parameters of the backbone neural network and the second prediction head of the neural network potential model according to the second measure of loss.

Additionally, in one or more implementations, the series of acts 700 can include generating feature representations by generating, utilizing the backbone neural network of the neural network potential model, a feature representation for a training compound geometry; generating, utilizing the first prediction head, the first quantum mechanics property prediction from the feature representation of the training compound geometry; and generating, utilizing the second prediction head, the second quantum mechanics property prediction from the feature representation of the training compound geometry.

Further, in some implementations, the series of acts 700 can include modifying parameters of the neural network potential model by comparing the first quantum mechanics property prediction with a first ground truth quantum mechanics property prediction for the training compound geometry from the first quantum mechanics representation class and the second quantum mechanics property prediction with a second ground truth quantum mechanics property prediction for the training compound geometry from the second quantum mechanics representation class.

Additionally, in one or more implementations, the series of acts 700 can include generating the first quantum mechanics property prediction from a first feature representation of a first training compound geometry; and generating the second quantum mechanics property prediction from a second feature representation of a second training compound geometry.

Moreover, in some implementations, the first quantum mechanics representation class corresponds to a high-fidelity quantum mechanics representation class and further comprising comparing the first quantum mechanics property prediction with the first ground truth from the high-fidelity quantum mechanics representation class, and the second quantum mechanics representation class corresponds to a low-fidelity quantum mechanics representation class having a lower measure of accuracy relative to the high-fidelity quantum mechanics representation class and further comprising comparing the second quantum mechanics property prediction with the second ground truth from the low-fidelity quantum mechanics representation class.

Further, in one or more implementations, the series of acts 700 can include generating, by a first quantum mechanics model, the first ground truth from the first quantum mechanics representation class; and generating, by a second quantum mechanics model, the second ground truth from the second quantum mechanics representation class.

Additionally, in some implementations, the series of acts 700 can include receiving a query compound geometry from a computing device, generating, by the backbone neural network of the neural network potential model, a feature representation of the query compound geometry; and generating, utilizing the first prediction head of the neural network potential model, a quantum mechanics property prediction according to the first quantum mechanics representation class for the query compound geometry from the feature representation.

FIG. 8 illustrates a block diagram of an example computing device 800 that may be configured to perform one or more of the processes described above. One will appreciate that one or more computing devices, such as the computing device 800 may represent the computing devices described above. In one or more embodiments, the computing device 800 may be a mobile device (e.g., a mobile telephone, a smartphone, a PDA, a tablet, a laptop, a camera, a tracker, a watch, a wearable device, etc.). In some embodiments, the computing device 800 may be a non-mobile device (e.g., a desktop computer or another type of client device). Further, the computing device 800 may be a server device that includes cloud-based processing and storage capabilities.

As shown in FIG. 8, the computing device 800 can include one or more processor(s) 802, memory 804, a storage device 806, input/output interfaces 808 (or “I/O interfaces 808”), and a communication interface 810, which may be communicatively coupled by way of a communication infrastructure (e.g., bus 812). While the computing device 800 is shown in FIG. 8, the components illustrated in FIG. 8 are not intended to be limiting. Additional or alternative components may be used in other embodiments. Furthermore, in certain embodiments, the computing device 800 includes fewer components than those shown in FIG. 8. Components of the computing device 800 shown in FIG. 8 will now be described in additional detail.

In particular embodiments, the processor(s) 802 includes hardware for executing instructions, such as those making up a computer program. As an example, and not by way of limitation, to execute instructions, the processor(s) 802 may retrieve (or fetch) the instructions from an internal register, an internal cache, memory 804, or a storage device 806 and decode and execute them.

The computing device 800 includes memory 804, which is coupled to the processor(s) 802. The memory 804 may be used for storing data, metadata, and programs for execution by the processor(s). The memory 804 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 804 may be internal or distributed memory.

The computing device 800 includes a storage device 806 includes storage for storing data or instructions. As an example, and not by way of limitation, the storage device 806 can include a non-transitory storage medium described above. The storage device 806 may include a hard disk drive (HDD), flash memory, a Universal Serial Bus (USB) drive or a combination these or other storage devices.

As shown, the computing device 800 includes one or more I/O interfaces 808, which are provided to allow a user to provide input to (such as user strokes), receive output from, and otherwise transfer data to and from the computing device 800. These I/O interfaces 808 may include a mouse, keypad or a keyboard, a touch screen, camera, optical scanner, network interface, modem, other known I/O devices or a combination of such I/O interfaces 808. The touch screen may be activated with a stylus or a finger.

The I/O interfaces 808 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 808 are configured to provide graphical data to a display for presentation to a user. The graphical data may be representative of one or more graphical user interfaces and/or any other graphical content as may serve a particular implementation.

The computing device 800 can further include a communication interface 810. The communication interface 810 can include hardware, software, or both. The communication interface 810 provides one or more interfaces for communication (such as, for example, packet-based communication) between the computing device and one or more other computing devices or one or more networks. As an example, and not by way of limitation, communication interface 810 may include a network interface controller (NIC) or network adapter for communicating with an Ethernet or other wire-based network or a wireless NIC (WNIC) or wireless adapter for communicating with a wireless network, such as a WI-FI. The computing device 800 can further include a bus 812. The bus 812 can include hardware, software, or both that connects components of computing device 800 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 800 can include a client device that includes a requester application or a web browser, such as MICROSOFT INTERNET EXPLORER, GOOGLE CHROME, or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 602 may include a variety of servers, sub-systems, programs, modules, logs, and data stores. In particular embodiments, the tech-bio exploration system 602 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 602 may also include suitable components such as network interfaces, security mechanisms, load balancers, failover servers, management-and-network-operations consoles, other suitable components, or any suitable combination thereof. In particular embodiments, the tech-bio exploration system 602 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 602 and one or more client devices. An action logger may be used to receive communications from a web server about a user's actions on or off the tech-bio exploration system 602. In conjunction with the action log, a third party-content-object log may be maintained of user exposures to third party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 602. A privacy setting of a user determines how particular information associated with a user can be shared. The authorization server may allow users to opt in to or opt out of having their actions logged by the tech-bio exploration system 602 or shared with other systems, such as, for example, by setting appropriate privacy settings. Third party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating, utilizing a backbone neural network of a neural network potential model, feature representations from training compound geometries;
generating, utilizing a first prediction head of the neural network potential model corresponding to a first quantum mechanics representation class, a first quantum mechanics property prediction from the feature representations;
generating, utilizing a second prediction head of the neural network potential model corresponding to a second quantum mechanics representation class, a second quantum mechanics property prediction from the feature representations;
modifying parameters of the neural network potential model by comparing the first quantum mechanics property prediction with a first ground truth from the first quantum mechanics representation class and the second quantum mechanics property prediction with a second ground truth from the second quantum mechanics representation class; and
in response to receiving a query compound geometry, generating, utilizing the first prediction head of the neural network potential model, a third quantum mechanics property prediction from the query compound geometry.

2. The computer-implemented method of claim 1, further comprising:
comparing, utilizing a loss function, the first quantum mechanics property prediction with the first ground truth from the first quantum mechanics representation class to determine a first measure of loss; and
modifying parameters of the backbone neural network and the first prediction head of the neural network potential model according to the first measure of loss.

3. The computer-implemented method of claim 2, further comprising:
comparing, utilizing the loss function, the second quantum mechanics property prediction with the second ground truth from the second quantum mechanics representation class to determine a second measure of loss; and
modifying parameters of the backbone neural network and the second prediction head of the neural network potential model according to the second measure of loss.

4. The computer-implemented method of claim 1, further comprising:
generating feature representations by generating, utilizing the backbone neural network of the neural network potential model, a feature representation for a training compound geometry;
generating, utilizing the first prediction head, the first quantum mechanics property prediction from the feature representation of the training compound geometry; and
generating, utilizing the second prediction head, the second quantum mechanics property prediction from the feature representation of the training compound geometry.

5. The computer-implemented method of claim 4, further comprising modifying parameters of the neural network potential model by comparing the first quantum mechanics property prediction with a first ground truth quantum mechanics property prediction for the training compound geometry from the first quantum mechanics representation class and the second quantum mechanics property prediction with a second ground truth quantum mechanics property prediction for the training compound geometry from the second quantum mechanics representation class.

6. The computer-implemented method of claim 1, further comprising:
generating the first quantum mechanics property prediction from a first feature representation of a first training compound geometry; and
generating the second quantum mechanics property prediction from a second feature representation of a second training compound geometry.

7. The computer-implemented method of claim 1,
wherein the first quantum mechanics representation class corresponds to a high-fidelity quantum mechanics representation class and further comprising comparing the first quantum mechanics property prediction with the first ground truth from the high-fidelity quantum mechanics representation class, and
wherein the second quantum mechanics representation class corresponds to a low-fidelity quantum mechanics representation class having a lower measure of accuracy relative to the high-fidelity quantum mechanics representation class and further comprising comparing the second quantum mechanics property prediction with the second ground truth from the low-fidelity quantum mechanics representation class.

8. The computer-implemented method of claim 1, further comprising:
generating, by a first quantum mechanics model, the first ground truth from the first quantum mechanics representation class; and
generating, by a second quantum mechanics model, the second ground truth from the second quantum mechanics representation class.

9. The computer-implemented method of claim 1, further comprising:
receiving a query compound geometry from a computing device,
generating, by the backbone neural network of the neural network potential model, a feature representation of the query compound geometry; and
generating, utilizing the first prediction head of the neural network potential model, a quantum mechanics property prediction according to the first quantum mechanics representation class for the query compound geometry from the feature representation.

10. A system comprising:
at least one processor; and at least one non-transitory computer-readable storage medium storing instructions that, when executed by the at least one processor to:
generate, utilizing a backbone neural network of a neural network potential model, feature representations from training compound geometries;
generate, utilizing a first prediction head of the neural network potential model corresponding to a first quantum mechanics representation class, a first quantum mechanics property prediction from the feature representations;
generate, utilizing a second prediction head of the neural network potential model corresponding to a second quantum mechanics representation class, a second quantum mechanics property prediction from the feature representations;
modify parameters of the neural network potential model by comparing the first quantum mechanics property prediction with a first ground truth from the first quantum mechanics representation class and the second quantum mechanics property prediction with a second ground truth from the second quantum mechanics representation class; and
in response to receiving a query compound geometry, generate, utilizing the first prediction head of the neural network potential model, a third quantum mechanics property prediction from the query compound geometry.

11. The system of claim 10, further comprising instructions that, when executed by the at least one processor, cause the system to:
compare, utilizing a loss function, the first quantum mechanics property prediction with the first ground truth from the first quantum mechanics representation class to determine a first measure of loss; and
modify parameters of the backbone neural network and the first prediction head of the neural network potential model according to the first measure of loss.

12. The system of claim 11, further comprising instructions that, when executed by the at least one processor, cause the system to:
compare, utilizing the loss function, the second quantum mechanics property prediction with the second ground truth from the second quantum mechanics representation class to determine a second measure of loss; and
modify parameters of the backbone neural network and the second prediction head of the neural network potential model according to the second measure of loss.

13. The system of claim 10, further comprising instructions that, when executed by the at least one processor, cause the system to:
generate feature representations by generating, utilizing the backbone neural network of the neural network potential model, a feature representation for a training compound geometry;
generate, utilizing the first prediction head, the first quantum mechanics property prediction from the feature representation of the training compound geometry; and
generate, utilizing the second prediction head, the second quantum mechanics property prediction from the feature representation of the training compound geometry.

14. The system of claim 10, further comprising instructions that, when executed by the at least one processor, cause the system to:
generate the first quantum mechanics property prediction from a first feature representation of a first training compound geometry; and
generate the second quantum mechanics property prediction from a second feature representation of a second training compound geometry.

15. The system of claim 10,
wherein the first quantum mechanics representation class corresponds to a high-fidelity quantum mechanics representation class and further comprising comparing the first quantum mechanics property prediction with the first ground truth from the high-fidelity quantum mechanics representation class, and
wherein the second quantum mechanics representation class corresponds to a low-fidelity quantum mechanics representation class having a lower measure of accuracy relative to the high-fidelity quantum mechanics representation class and further comprising comparing the second quantum mechanics property prediction with the second ground truth from the low-fidelity quantum mechanics representation class.

16. A non-transitory computer-readable medium storing instructions that, when executed by at least one processor, cause a computing device to:
generate, utilizing a backbone neural network of a neural network potential model, feature representations from training compound geometries;
generate, utilizing a first prediction head of the neural network potential model corresponding to a first quantum mechanics representation class, a first quantum mechanics property prediction from the feature representations;
generate, utilizing a second prediction head of the neural network potential model corresponding to a second quantum mechanics representation class, a second quantum mechanics property prediction from the feature representations;
modify parameters of the neural network potential model by comparing the first quantum mechanics property prediction with a first ground truth from the first quantum mechanics representation class and the second quantum mechanics property prediction with a second ground truth from the second quantum mechanics representation class; and
in response to receiving a query compound geometry, generate, utilizing the first prediction head of the neural network potential model, a third quantum mechanics property prediction from the query compound geometry.

17. The non-transitory computer-readable medium of claim 16, further comprising instructions that, when executed by the at least one processor, cause the computing device to:
compare, utilizing a loss function, the first quantum mechanics property prediction with the first ground truth from the first quantum mechanics representation class to determine a first measure of loss; and
modify parameters of the backbone neural network and the first prediction head of the neural network potential model according to the first measure of loss.

18. The non-transitory computer-readable medium of claim 16, further comprising instructions that, when executed by the at least one processor, cause the computing device to:
generate feature representations by generating, utilizing the backbone neural network of the neural network potential model, a feature representation for a training compound geometry;
generate, utilizing the first prediction head, the first quantum mechanics property prediction from the feature representation of the training compound geometry; and
generate, utilizing the second prediction head, the second quantum mechanics property prediction from the feature representation of the training compound geometry.

19. The non-transitory computer-readable medium of claim 16, further comprising instructions that, when executed by the at least one processor, cause the computing device to:
generate the first quantum mechanics property prediction from a first feature representation of a first training compound geometry; and
generate the second quantum mechanics property prediction from a second feature representation of a second training compound geometry.

20. The non-transitory computer-readable medium of claim 16,
wherein the first quantum mechanics representation class corresponds to a high-fidelity quantum mechanics representation class and further comprising comparing the first quantum mechanics property prediction with the first ground truth from the high-fidelity quantum mechanics representation class, and
wherein the second quantum mechanics representation class corresponds to a low-fidelity quantum mechanics representation class having a lower measure of accuracy relative to the high-fidelity quantum mechanics representation class and further comprising comparing the second quantum mechanics property prediction with the second ground truth from the low-fidelity quantum mechanics representation class.
